SEQUENCE LISTING (140 sequences)

SEQ ID NO: 1 - RNA, 80 nt, synthetic construct
  source 1..80: mol_type = other RNA
gttttagagctagaaatagcaagttaaaataaggctagtccgttatcaacttgaaaaagtggcaccgagtcggtgctttt

SEQ ID NO: 2 - DNA, 41 bp, synthetic construct
  source 1..41: mol_type = other DNA
aattcgccagtgatgcaaaacgagggttgtcaaagacccca

SEQ ID NO: 3 - DNA, 41 bp, synthetic construct
  source 1..41: mol_type = other DNA
cagccattgatgtcgaaaaacgagggttgtcaaagacccca

SEQ ID NO: 4 - DNA, 41 bp, synthetic construct
  source 1..41: mol_type = other DNA
ggccgctagtaattcaaaaacgagggttgtcaaagacccca

SEQ ID NO: 5 - DNA, 41 bp, synthetic construct
  source 1..41: mol_type = other DNA
tcaggcgccgattaataaaacgagggttgtcaaagacccca

SEQ ID NO: 6 - DNA, 41 bp, synthetic construct
  source 1..41: mol_type = other DNA
actaagcgaggtctctaaaacgagggttgtcaaagacccca

SEQ ID NO: 7 - DNA, 41 bp, synthetic construct
  source 1..41: mol_type = other DNA
cgttccatcgagtaagaaaacgagggttgtcaaagacccca

SEQ ID NO: 8 - DNA, 41 bp, synthetic construct
  source 1..41: mol_type = other DNA
actgttcagacgatcgaaaacgagggttgtcaaagacccca

SEQ ID NO: 9 - DNA, 41 bp, synthetic construct
  source 1..41: mol_type = other DNA
cctcgacgtggataataaaacgagggttgtcaaagacccca

SEQ ID NO: 10 - DNA, 41 bp, synthetic construct
  source 1..41: mol_type = other DNA
gtacatcggatgatccaaaacgagggttgtcaaagacccca

SEQ ID NO: 11 - DNA, 41 bp, synthetic construct
  source 1..41: mol_type = other DNA
tacggcgctattgaacaaaacgagggttgtcaaagacccca

SEQ ID NO: 12 - DNA, 41 bp, synthetic construct
  source 1..41: mol_type = other DNA
aattcgccagtgatgcgagggaagtggcactgagcaaatct

SEQ ID NO: 13 - DNA, 41 bp, synthetic construct
  source 1..41: mol_type = other DNA
cagccattgatgtcgagagggaagtggcactgagcaaatct

SEQ ID NO: 14 - DNA, 41 bp, synthetic construct
  source 1..41: mol_type = other DNA
ggccgctagtaattcagagggaagtggcactgagcaaatct

SEQ ID NO: 15 - DNA, 41 bp, synthetic construct
  source 1..41: mol_type = other DNA
tcaggcgccgattaatgagggaagtggcactgagcaaatct

SEQ ID NO: 16 - DNA, 41 bp, synthetic construct
  source 1..41: mol_type = other DNA
actaagcgaggtctctgagggaagtggcactgagcaaatct

SEQ ID NO: 17 - DNA, 41 bp, synthetic construct
  source 1..41: mol_type = other DNA
cgttccatcgagtaaggagggaagtggcactgagcaaatct

SEQ ID NO: 18 - DNA, 41 bp, synthetic construct
  source 1..41: mol_type = other DNA
actgttcagacgatcggagggaagtggcactgagcaaatct

SEQ ID NO: 19 - DNA, 41 bp, synthetic construct
  source 1..41: mol_type = other DNA
cctcgacgtggataatgagggaagtggcactgagcaaatct

SEQ ID NO: 20 - DNA, 41 bp, synthetic construct
  source 1..41: mol_type = other DNA
gtacatcggatgatccgagggaagtggcactgagcaaatct

SEQ ID NO: 21 - DNA, 41 bp, synthetic construct
  source 1..41: mol_type = other DNA
tacggcgctattgaacgagggaagtggcactgagcaaatct

SEQ ID NO: 22 - DNA, 57 bp, synthetic construct
  source 1..57: mol_type = other DNA
cgtcgttaacagcgtacagccattgatgtcgaaaagtcccgatgatccattgcctcc

SEQ ID NO: 23 - DNA, 57 bp, synthetic construct
  source 1..57: mol_type = other DNA
cgatagtcttacgagcggccgctagtaattcaaaagtcccgatgatccattgcctcc

SEQ ID NO: 24 - DNA, 57 bp, synthetic construct
  source 1..57: mol_type = other DNA
gcgaacgatcagtctttcaggcgccgattaataaagtcccgatgatccattgcctcc

SEQ ID NO: 25 - DNA, 57 bp, synthetic construct
  source 1..57: mol_type = other DNA
tgccatggcgtatacaactaagcgaggtctctaaagtcccgatgatccattgcctcc

SEQ ID NO: 26 - DNA, 57 bp, synthetic construct
  source 1..57: mol_type = other DNA
agcgcatcattggcatcgttccatcgagtaagaaagtcccgatgatccattgcctcc

SEQ ID NO: 27 - DNA, 57 bp, synthetic construct
  source 1..57: mol_type = other DNA
gacgacgtatgtacctaattcgccagtgatgcaaagtcccgatgatccattgcctcc

SEQ ID NO: 28 - DNA, 57 bp, synthetic construct
  source 1..57: mol_type = other DNA
ataagttgcgcacgctactgttcagacgatcgaaagtcccgatgatccattgcctcc

SEQ ID NO: 29 - DNA, 57 bp, synthetic construct
  source 1..57: mol_type = other DNA
ataacacggtccggttcctcgacgtggataataaagtcccgatgatccattgcctcc

SEQ ID NO: 30 - DNA, 57 bp, synthetic construct
  source 1..57: mol_type = other DNA
ggttagattcacgaccgtacatcggatgatccaaagtcccgatgatccattgcctcc

SEQ ID NO: 31 - DNA, 57 bp, synthetic construct
  source 1..57: mol_type = other DNA
aacggttcatgagccttacggcgctattgaacaaagtcccgatgatccattgcctcc

SEQ ID NO: 32 - DNA, 58 bp, synthetic construct
  source 1..58: mol_type = other DNA
cgtcgttaacagcgtacagccattgatgtcgaacaaacctgataacgcaagctactgc

SEQ ID NO: 33 - DNA, 58 bp, synthetic construct
  source 1..58: mol_type = other DNA
cgatagtcttacgagcggccgctagtaattcaacaaacctgataacgcaagctactgc

SEQ ID NO: 34 - DNA, 58 bp, synthetic construct
  source 1..58: mol_type = other DNA
gcgaacgatcagtctttcaggcgccgattaatacaaacctgataacgcaagctactgc

SEQ ID NO: 35 - DNA, 58 bp, synthetic construct
  source 1..58: mol_type = other DNA
tgccatggcgtatacaactaagcgaggtctctacaaacctgataacgcaagctactgc

SEQ ID NO: 36 - DNA, 58 bp, synthetic construct
  source 1..58: mol_type = other DNA
agcgcatcattggcatcgttccatcgagtaagacaaacctgataacgcaagctactgc

SEQ ID NO: 37 - DNA, 58 bp, synthetic construct
  source 1..58: mol_type = other DNA
gacgacgtatgtacctaattcgccagtgatgcacaaacctgataacgcaagctactgc

SEQ ID NO: 38 - DNA, 58 bp, synthetic construct
  source 1..58: mol_type = other DNA
ataagttgcgcacgctactgttcagacgatcgacaaacctgataacgcaagctactgc

SEQ ID NO: 39 - DNA, 58 bp, synthetic construct
  source 1..58: mol_type = other DNA
ataacacggtccggttcctcgacgtggataatacaaacctgataacgcaagctactgc

SEQ ID NO: 40 - DNA, 58 bp, synthetic construct
  source 1..58: mol_type = other DNA
aacggttcatgagccttacggcgctattgaacacaaacctgataacgcaagctactgc

SEQ ID NO: 41 - DNA, 58 bp, synthetic construct
  source 1..58: mol_type = other DNA
ggttagattcacgaccgtacatcggatgatccacaaacctgataacgcaagctactgc

SEQ ID NO: 42 - DNA, 57 bp, synthetic construct
  source 1..57: mol_type = other DNA
tcgtatcgtgagcgtcaaccgactgagcataaaaaacgagggttgtcaaagacccca

SEQ ID NO: 43 - DNA, 57 bp, synthetic construct
  source 1..57: mol_type = other DNA
ttagtcactgtacagcgtgaggcgtagttcacaaaacgagggttgtcaaagacccca

SEQ ID NO: 44 - DNA, 57 bp, synthetic construct
  source 1..57: mol_type = other DNA
atcagtacgttgctagcttgagcgatagccagaaaacgagggttgtcaaagacccca

SEQ ID NO: 45 - DNA, 57 bp, synthetic construct
  source 1..57: mol_type = other DNA
agtatgcacgaccggatctgtcaacgatacgtaaaacgagggttgtcaaagacccca

SEQ ID NO: 46 - DNA, 57 bp, synthetic construct
  source 1..57: mol_type = other DNA
gaagtctagatcaatcgttagcatctgctcgcaaaacgagggttgtcaaagacccca

SEQ ID NO: 47 - DNA, 57 bp, synthetic construct
  source 1..57: mol_type = other DNA
agacgtctgacgatgctcataacctggacatcaaaacgagggttgtcaaagacccca

SEQ ID NO: 48 - DNA, 57 bp, synthetic construct
  source 1..57: mol_type = other DNA
ctcattcgatgtatgtcgcaggtagcaagcataaaacgagggttgtcaaagacccca

SEQ ID NO: 49 - DNA, 57 bp, synthetic construct
  source 1..57: mol_type = other DNA
tactgtcgattcgaccacgactaggctatgctaaaacgagggttgtcaaagacccca

SEQ ID NO: 50 - DNA, 57 bp, synthetic construct
  source 1..57: mol_type = other DNA
ctgtactccgatgaacggcgatctagtctacgaaaacgagggttgtcaaagacccca

SEQ ID NO: 51 - DNA, 57 bp, synthetic construct
  source 1..57: mol_type = other DNA
cgatggtactcagatcggcgacatcagttgataaaacgagggttgtcaaagacccca

SEQ ID NO: 52 - DNA, 57 bp, synthetic construct
  source 1..57: mol_type = other DNA
agtgctagtcgatgccgctgcatacctatgataaaacgagggttgtcaaagacccca

SEQ ID NO: 53 - DNA, 57 bp, synthetic construct
  source 1..57: mol_type = other DNA
acatcttacggctcgactggcacgatgtctgaaaaacgagggttgtcaaagacccca

SEQ ID NO: 54 - DNA, 57 bp, synthetic construct
  source 1..57: mol_type = other DNA
acttaagtcgagtcgcatgcctgtcgctagataaaacgagggttgtcaaagacccca

SEQ ID NO: 55 - DNA, 57 bp, synthetic construct
  source 1..57: mol_type = other DNA
atcaagatgtaccacgtcaggctagtactgctaaaacgagggttgtcaaagacccca

SEQ ID NO: 56 - DNA, 57 bp, synthetic construct
  source 1..57: mol_type = other DNA
tcgagcttcgagtgataacgtaacgctgcgtaaaaacgagggttgtcaaagacccca

SEQ ID NO: 57 - DNA, 57 bp, synthetic construct
  source 1..57: mol_type = other DNA
gcagatgaccactacgtcgaactgacttgactaaaacgagggttgtcaaagacccca

SEQ ID NO: 58 - DNA, 57 bp, synthetic construct
  source 1..57: mol_type = other DNA
tcagcatagcgtcgatcaccaatgcatgctagaaaacgagggttgtcaaagacccca

SEQ ID NO: 59 - DNA, 57 bp, synthetic construct
  source 1..57: mol_type = other DNA
ctcgatgacagatgcgatactggcgttcaatgaaaacgagggttgtcaaagacccca

SEQ ID NO: 60 - DNA, 57 bp, synthetic construct
  source 1..57: mol_type = other DNA
gcgtcagctacgattgtatccaagtgctcgataaaacgagggttgtcaaagacccca

SEQ ID NO: 61 - DNA, 57 bp, synthetic construct
  source 1..57: mol_type = other DNA
gacattgactgctatgacgccttgagtagcagaaaacgagggttgtcaaagacccca

SEQ ID NO: 62 - DNA, 57 bp, synthetic construct
  source 1..57: mol_type = other DNA
agcaacgctagtggccgctatgtactagctcgaaaacgagggttgtcaaagacccca

SEQ ID NO: 63 - DNA, 57 bp, synthetic construct
  source 1..57: mol_type = other DNA
tacatctggcgagtatgatcctacggtgagtcaaaacgagggttgtcaaagacccca

SEQ ID NO: 64 - DNA, 57 bp, synthetic construct
  source 1..57: mol_type = other DNA
atcagctgttacgataggcgactcgccatcgaaaaacgagggttgtcaaagacccca

SEQ ID NO: 65 - DNA, 57 bp, synthetic construct
  source 1..57: mol_type = other DNA
cgcactagtatcagcctaagcactcgtgatggaaaacgagggttgtcaaagacccca

SEQ ID NO: 66 - DNA, 58 bp, synthetic construct
  source 1..58: mol_type = other DNA
tcgtatcgtgagcgtcaaccgactgagcataaacaaacctgataacgcaagctactgc

SEQ ID NO: 67 - DNA, 58 bp, synthetic construct
  source 1..58: mol_type = other DNA
ttagtcactgtacagcgtgaggcgtagttcacacaaacctgataacgcaagctactgc

SEQ ID NO: 68 - DNA, 58 bp, synthetic construct
  source 1..58: mol_type = other DNA
atcagtacgttgctagcttgagcgatagccagacaaacctgataacgcaagctactgc

SEQ ID NO: 69 - DNA, 58 bp, synthetic construct
  source 1..58: mol_type = other DNA
agtatgcacgaccggatctgtcaacgatacgtacaaacctgataacgcaagctactgc

SEQ ID NO: 70 - DNA, 58 bp, synthetic construct
  source 1..58: mol_type = other DNA
gaagtctagatcaatcgttagcatctgctcgcacaaacctgataacgcaagctactgc

SEQ ID NO: 71 - DNA, 58 bp, synthetic construct
  source 1..58: mol_type = other DNA
agacgtctgacgatgctcataacctggacatcacaaacctgataacgcaagctactgc

SEQ ID NO: 72 - DNA, 58 bp, synthetic construct
  source 1..58: mol_type = other DNA
ctcattcgatgtatgtcgcaggtagcaagcatacaaacctgataacgcaagctactgc

SEQ ID NO: 73 - DNA, 58 bp, synthetic construct
  source 1..58: mol_type = other DNA
tactgtcgattcgaccacgactaggctatgctacaaacctgataacgcaagctactgc

SEQ ID NO: 74 - DNA, 58 bp, synthetic construct
  source 1..58: mol_type = other DNA
ctgtactccgatgaacggcgatctagtctacgacaaacctgataacgcaagctactgc

SEQ ID NO: 75 - DNA, 58 bp, synthetic construct
  source 1..58: mol_type = other DNA
cgatggtactcagatcggcgacatcagttgatacaaacctgataacgcaagctactgc

SEQ ID NO: 76 - DNA, 58 bp, synthetic construct
  source 1..58: mol_type = other DNA
agtgctagtcgatgccgctgcatacctatgatacaaacctgataacgcaagctactgc

SEQ ID NO: 77 - DNA, 58 bp, synthetic construct
  source 1..58: mol_type = other DNA
acatcttacggctcgactggcacgatgtctgaacaaacctgataacgcaagctactgc

SEQ ID NO: 78 - DNA, 58 bp, synthetic construct
  source 1..58: mol_type = other DNA
acttaagtcgagtcgcatgcctgtcgctagatacaaacctgataacgcaagctactgc

SEQ ID NO: 79 - DNA, 58 bp, synthetic construct
  source 1..58: mol_type = other DNA
atcaagatgtaccacgtcaggctagtactgctacaaacctgataacgcaagctactgc

SEQ ID NO: 80 - DNA, 58 bp, synthetic construct
  source 1..58: mol_type = other DNA
tcgagcttcgagtgataacgtaacgctgcgtaacaaacctgataacgcaagctactgc

SEQ ID NO: 81 - DNA, 58 bp, synthetic construct
  source 1..58: mol_type = other DNA
gcagatgaccactacgtcgaactgacttgactacaaacctgataacgcaagctactgc

SEQ ID NO: 82 - DNA, 58 bp, synthetic construct
  source 1..58: mol_type = other DNA
tcagcatagcgtcgatcaccaatgcatgctagacaaacctgataacgcaagctactgc

SEQ ID NO: 83 - DNA, 58 bp, synthetic construct
  source 1..58: mol_type = other DNA
ctcgatgacagatgcgatactggcgttcaatgacaaacctgataacgcaagctactgc

SEQ ID NO: 84 - DNA, 58 bp, synthetic construct
  source 1..58: mol_type = other DNA
gcgtcagctacgattgtatccaagtgctcgatacaaacctgataacgcaagctactgc

SEQ ID NO: 85 - DNA, 58 bp, synthetic construct
  source 1..58: mol_type = other DNA
gacattgactgctatgacgccttgagtagcagacaaacctgataacgcaagctactgc

SEQ ID NO: 86 - DNA, 58 bp, synthetic construct
  source 1..58: mol_type = other DNA
agcaacgctagtggccgctatgtactagctcgacaaacctgataacgcaagctactgc

SEQ ID NO: 87 - DNA, 58 bp, synthetic construct
  source 1..58: mol_type = other DNA
tacatctggcgagtatgatcctacggtgagtcacaaacctgataacgcaagctactgc

SEQ ID NO: 88 - DNA, 58 bp, synthetic construct
  source 1..58: mol_type = other DNA
atcagctgttacgataggcgactcgccatcgaacaaacctgataacgcaagctactgc

SEQ ID NO: 89 - DNA, 58 bp, synthetic construct
  source 1..58: mol_type = other DNA
cgcactagtatcagcctaagcactcgtgatggacaaacctgataacgcaagctactgc

SEQ ID NO: 90 - DNA, 20 bp, synthetic construct
  source 1..20: mol_type = other DNA
ttgggagggtcctcacagta

SEQ ID NO: 91 - DNA, 20 bp, synthetic construct
  source 1..20: mol_type = other DNA
gaggttgcagtgagccaaga

SEQ ID NO: 92 - DNA, 21 bp, synthetic construct
  source 1..21: mol_type = other DNA
ccgccgccgccgccgccgccg

SEQ ID NO: 93 - DNA, 30 bp, synthetic construct
  source 1..30: mol_type = other DNA
ccgccgccgccgccgccgccgccgccgccg

SEQ ID NO: 94 - DNA, 17 bp, synthetic construct
  source 1..17: mol_type = other DNA
gctccaggcgtcggcgg

SEQ ID NO: 95 - DNA, 21 bp, synthetic construct
  source 1..21: mol_type = other DNA
gccccgctccaggcgtcggcg

SEQ ID NO: 96 - DNA, 52 bp, synthetic construct
  source 1..52: mol_type = other DNA
gtcgccccgctccaggcgtcggcgggggatcctttccgcatgggcctgcgcc

SEQ ID NO: 97 - DNA, 52 bp, synthetic construct
  source 1..52: mol_type = other DNA
ggcgcaggcccatgcggaaaggatcccccgccgacgcctggagcggggcgac

SEQ ID NO: 98 - DNA, 52 bp, synthetic construct
  source 1..52: mol_type = other DNA
gtcgccccgctccaggcgtcggcggggcatcctttccgcatgggcctgcgcc

SEQ ID NO: 99 - DNA, 52 bp, synthetic construct
  source 1..52: mol_type = other DNA
ggcgcaggcccatgcggaaaggatgccccgccgacgcctggagcggggcgac

SEQ ID NO: 100 - DNA, 19 bp, synthetic construct
  source 1..19: mol_type = other DNA
ggcgcggggctcaacggag

SEQ ID NO: 101 - DNA, 49 bp, synthetic construct
  source 1..49: mol_type = other DNA
ttacagtctcaccacgccccgtcccctctccgttgagccccgcgccttc

SEQ ID NO: 102 - DNA, 49 bp, synthetic construct
  source 1..49: mol_type = other DNA
gaaggcgcggggctcaacggagaggggacggggcgtggtgagactgtaa

SEQ ID NO: 103 - DNA, 49 bp, synthetic construct
  source 1..49: mol_type = other DNA
ttacagtctcaccacgccccgtccgctctccgttgagccccgcgccttc

SEQ ID NO: 104 - DNA, 49 bp, synthetic construct
  source 1..49: mol_type = other DNA
gaaggcgcggggctcaacggagagcggacggggcgtggtgagactgtaa

SEQ ID NO: 105 - DNA, 20 bp, synthetic construct
  source 1..20: mol_type = other DNA
gtctgggacgcaaggcgccg

SEQ ID NO: 106 - DNA, 49 bp, synthetic construct
  source 1..49: mol_type = other DNA
gcgtctgggacgcaaggcgccgtgggggctgccgggacgggtccaagat

SEQ ID NO: 107 - DNA, 49 bp, synthetic construct
  source 1..49: mol_type = other DNA
atcttggacccgtcccggcagcccccacggcgccttgcgtcccagacgc

SEQ ID NO: 108 - DNA, 49 bp, synthetic construct
  source 1..49: mol_type = other DNA
gcgtctgggacgcaaggcgccgtgagggctgccgggacgggtccaagat

SEQ ID NO: 109 - DNA, 49 bp, synthetic construct
  source 1..49: mol_type = other DNA
atcttggacccgtcccggcagccctcacggcgccttgcgtcccagacgc

SEQ ID NO: 110 - DNA, 19 bp, synthetic construct
  source 1..19: mol_type = other DNA
gatgcacgcggggtggggc

SEQ ID NO: 111 - DNA, 49 bp, synthetic construct
  source 1..49: mol_type = other DNA
tctgcgtcagggtttccttcttttccagccccaccccgcgtgcatccca

SEQ ID NO: 112 - DNA, 49 bp, synthetic construct
  source 1..49: mol_type = other DNA
tgggatgcacgcggggtggggctggaaaagaaggaaaccctgacgcaga

SEQ ID NO: 113 - DNA, 49 bp, synthetic construct
  source 1..49: mol_type = other DNA
tctgcgtcagggtttccttcttttgcagccccaccccgcgtgcatccca

SEQ ID NO: 114 - DNA, 49 bp, synthetic construct
  source 1..49: mol_type = other DNA
tgggatgcacgcggggtggggctgcaaaagaaggaaaccctgacgcaga

SEQ ID NO: 115 - DNA, 17 bp, synthetic construct
  source 1..17: mol_type = other DNA
attcaggttgatgtcct

SEQ ID NO: 116 - DNA, 20 bp, synthetic construct
  source 1..20: mol_type = other DNA
atcccattctgaggttctgg

SEQ ID NO: 117 - DNA, 49 bp, synthetic construct
  source 1..49: mol_type = other DNA
ccctcattcaggttgatgtcctaagccccagaacctcagaatgggattg

SEQ ID NO: 118 - DNA, 49 bp, synthetic construct
  source 1..49: mol_type = other DNA
caatcccattctgaggttctggggcttaggacatcaacctgaatgaggg

SEQ ID NO: 119 - DNA, 49 bp, synthetic construct
  source 1..49: mol_type = other DNA
ccctcattcaggttgatgtcctaacccccagaacctcagaatgggattg

SEQ ID NO: 120 - DNA, 49 bp, synthetic construct
  source 1..49: mol_type = other DNA
caatcccattctgaggttctgggggttaggacatcaacctgaatgaggg

SEQ ID NO: 121 - DNA, 17 bp, synthetic construct
  source 1..17: mol_type = other DNA
gcaggcagagaggagcc

SEQ ID NO: 122 - DNA, 19 bp, synthetic construct
  source 1..19: mol_type = other DNA
gcctggctaaagtaggctt

SEQ ID NO: 123 - DNA, 49 bp, synthetic construct
  source 1..49: mol_type = other DNA
gtggcctggctaaagtaggctttactgggctcctctctgcctgcatcac

SEQ ID NO: 124 - DNA, 49 bp, synthetic construct
  source 1..49: mol_type = other DNA
gtgatgcaggcagagaggagcccagtaaagcctactttagccaggccac

SEQ ID NO: 125 - DNA, 49 bp, synthetic construct
  source 1..49: mol_type = other DNA
gtggcctggctaaagtaggctttagtgggctcctctctgcctgcatcac

SEQ ID NO: 126 - DNA, 49 bp, synthetic construct
  source 1..49: mol_type = other DNA
gtgatgcaggcagagaggagcccactaaagcctactttagccaggccac

SEQ ID NO: 127 - DNA, 18 bp, synthetic construct
  source 1..18: mol_type = other DNA
gcttttaggacgcctcgg

SEQ ID NO: 128 - DNA, 21 bp, synthetic construct
  source 1..21: mol_type = other DNA
gtgcttttaggacgcctcggc

SEQ ID NO: 129 - DNA, 48 bp, synthetic construct
  source 1..48: mol_type = other DNA
aatgcttttaggacgcctcggcgggagtggcggggcagggggggggcg

SEQ ID NO: 130 - DNA, 48 bp, synthetic construct
  source 1..48: mol_type = other DNA
cgccccccccctgccccgccactcccgccgaggcgtcctaaaagcatt

SEQ ID NO: 131 - DNA, 18 bp, synthetic construct
  source 1..18: mol_type = other DNA
gcgggacacttcgagagg

SEQ ID NO: 132 - DNA, 48 bp, synthetic construct
  source 1..48: mol_type = other DNA
ggcgcgggacacttcgagaggaggcggggtttggagctggagagatgt

SEQ ID NO: 133 - DNA, 48 bp, synthetic construct
  source 1..48: mol_type = other DNA
acatctctccagctccaaaccccgcctcctctcgaagtgtcccgcgcc

SEQ ID NO: 134 - DNA, 22 bp, synthetic construct
  source 1..22: mol_type = other DNA
gatagggaaatgtcagggttaa

SEQ ID NO: 135 - DNA, 51 bp, synthetic construct
  source 1..51: mol_type = other DNA
ttaaaaataaaaataagttaacactcgattaaccctgacatttccctatcc

SEQ ID NO: 136 - DNA, 51 bp, synthetic construct
  source 1..51: mol_type = other DNA
ggatagggaaatgtcagggttaatcgagtgttaacttatttttatttttaa

SEQ ID NO: 137 - DNA, 51 bp, synthetic construct
  source 1..51: mol_type = other DNA
ttaaaaataaaaataagttaacacttgattaaccctgacatttccctatcc

SEQ ID NO: 138 - DNA, 51 bp, synthetic construct
  source 1..51: mol_type = other DNA
ggatagggaaatgtcagggttaatcaagtgttaacttatttttatttttaa

SEQ ID NO: 139 - DNA, 22 bp, synthetic construct
  source 1..22: mol_type = other DNA
gccccgctccaggcgtcggcgg

SEQ ID NO: 140 - DNA, 23 bp, synthetic construct
  source 1..23: mol_type = other DNA
taaaaataaaaataagttaacac

(19) United States
(12) Patent Application Publication
(10) Pub. No.: US 2026/0327584 A1
(43) Pub. Date: Oct. 22, 2026

(54) COMPOSITIONS AND METHODS FOR TREATING HUNTINGTON'S DISEASE AND RELATED DISORDERS

(71) Applicant: The Children's Hospital of Philadelphia, Philadelphia, PA (US)

(72) Inventors: Beverly L. Davidson, Philadelphia, PA (US); Alejandro Monteys, Ballerup (DK); Kai Wang, Princeton, NJ (US); Li Fang, Guangdong Province (CN)

(21) Appl. No.: 19/105,889

(22) PCT Filed: Sep. 25, 2023
(86) PCT No.: PCT/US2023/075008

§ 371 Date: Feb. 24, 2025

Related U.S. Application Data
(60) Provisional application No. 63/409,409, filed on Sep. 23, 2022.

(51) Int. Cl. C12N 9/22; A61K 48/00; C07K 14/47; C12N 15/11

(52) U.S. Cl. CPC C12N 9/226; A61K 48/005; C07K 14/47; C12N 15/111; C12N 2310/20; C12N 2320/34

(57) ABSTRACT

Compositions and methods are provided for the inhibition, treatment and/or prevention of Huntington's disease and related disorders.

This application claims priority under 35 U.S.C. § 119 (e) to U.S. Provisional Patent Application No. 63/409,409, filed Sep. 23, 2022. The foregoing application is incorporated by reference herein.

### FIELD OF THE INVENTION

The present invention relates to polyglutamine (polyQ) disorders, particularly Huntington's disease. Specifically, the instant invention provides compositions and methods for the treatment and/or prevention of Huntington's disease and related disorders.

### BACKGROUND OF THE INVENTION

Huntington's disease (HD, MIM: 143100 (omim.org)) is an autosomal dominant neurodegenerative disease that affects 10.6-13.7 individuals per 100,000 in populations of European ancestry (The Huntington's Disease Collaborative Research Group (1993) Cell 72:971-983; Bates, et al. (2015) Nat. Rev. Dis. Primers 1:15005; McColgan, et al. (2018) Eur. J. Neurol., 25:24-34; Fisher, et al. (2014) Mov. Disord., 29:105-114). Individuals with HD suffer from progressive motor, cognitive and psychiatric disturbances over the course of 10-20 years (Roos, R. A. (2010) Orphanet. J. Rare Dis., 5:40; Labbadia, et al. (2013) Trends Biochem. Sci., 38:378-385). At the molecular level, HD is caused by a CAG trinucleotide repeat expansion in exon-1 of the huntingtin (HTT, MIM: 613004) gene located at chromosome 4p16.3. In the normal population, the CAG repeat is in the range of 6-35. When expanded to ≥35 repeats, HD is likely to develop. In individuals with 36-39 repeats, there is partial penetrance with full penetrance when there are ≥40 repeats (Bates, et al. (2015) Nat. Rev. Dis. Primers 1:15005). The predicted rate by which an individual with CAG expansion will develop HD is determined by the longest expanded allele in a completely dominant manner (Lee, et al. (2012) Neurology 78:690-695).

Currently, there is only symptomatic treatment for individuals with HD (Bates, et al. (2015) Nat. Rev. Dis. Primers 1:15005; Johnson et al. (2010) Hum. Mol. Genet., 19: R98-R102). Earlier studies using mouse models showed that HD-like phenotypes can be resolved if the expression of the expanded HTT (mHTT) allele is reduced, even at later disease stages, indicating that reducing HTT expression, and in particular expanded HTT expression, may be clinically relevant (Yamamoto, et al. (2000) Cell 101:57-66; Diaz-Hernandez et al. (2005) J. Neurosci., 25:9773-9781 Harper, et al. (2005) Proc. Natl. Acad. Sci., 102:5820-5825; DiFiglia, et al. (2007) Proc. Natl. Acad. Sci., 104:17204-17209).

Gene silencing strategies using RNA interference (RNAi) or antisense oligonucleotides (ASO), have been efficacious in mouse models (Harper, et al. (2005) Proc. Natl. Acad. Sci., 102:5820-5825; Kordasiewicz, et al. (2012) Neuron 74:1031-1044). One trial done in collaboration between Ionis Pharmaceuticals and Roche approached non-allele selective silencing for HD individuals (Tabrizi, et al. (2019) N. Engl. J. Med., 380:2307-2316; ClinicalTrials.gov Identifiers: NCT02519036, and NCT03342053). Phase III studies were ended early because participants receiving active drug progressed more rapidly than placebo treated participants (ClinicalTrials.gov Identifier: NCT03761849). Additionally, two allele-selective ASOs were tested in individuals with HD (PRECISION-HD1 and PRECISION-HD2; ClinicalTrials.gov Identifiers: NCT03225833 and NCT03225846) with results released recently. Unfortunately, neither ASO showed target engagement in cerebrospinal fluid (CSF). The PRECISION-HD2 core trial participants who received WVE-120102 (targeting rs362331) had a median reduction of 9.9% in mHTT in CSF (p=0.74) compared to the placebo group who had a median decrease in mHTT of 0.8%. Results of the PRECISION-HD1 core trial were similar. Thus, it is important to develop and test additional gene silencing strategies.

### SUMMARY OF THE INVENTION

In accordance with one aspect of the instant invention, methods and compositions for inhibiting, treating, and/or preventing a polyglutamine disorder (e.g., Huntington's disease) in a subject are provided. In accordance with another aspect of the instant invention, methods for reducing the expression of a mutant protein (e.g., mutant huntingtin) encoded by an allele of a gene associated with a polyglutamine disorder (e.g., Huntington's disease) in a cell are provided. In a particular embodiment, the methods of the instant invention comprise administering to the subject or cell Cas9 or a nucleic acid molecule encoding Cas9 and at least one guide RNA (e.g., sgRNA). In a particular embodiment, the guide RNA are administered to the subject or cell as a nucleic acid molecule (e.g., an expression vector or viral vector) encoding the guide RNA. In a particular embodiment, the methods of the instant invention comprise administering two guide RNAs to the subject or cell. In a particular embodiment, one guide RNA targets a sequence 5′ of exon 1 (e.g., within the 5′ untranslated region, within the promoter, or within the first 2 kb 5′ of the transcription start site) and one guide RNA targets a sequence within intron 1. In a particular embodiment, at least one guide RNA administered to the subject or cell targets a sequence adjacent to a PAM present on only one allele of the gene (e.g., the mutant allele). In a particular embodiment, at least one of the guide RNAs targets a sequence specifically set forth herein.

The instant invention also encompasses guide RNAs, nucleic acid molecules (e.g., an expression vector or viral vector) encoding the guide RNAs, and compositions comprising the guide RNAs and/or nucleic acid molecules (e.g., an expression vector or viral vector) encoding the guide RNAs. In a particular embodiment, the composition and nucleic acid molecules (e.g., an expression vector or viral vector) encoding the guide RNAs contain or encode more than one guide RNA.

In accordance with another aspect of the instant invention, methods of identifying target sequences for gRNA are provided. In a particular embodiment, the methods provide for the identification of guide RNAs for allele-specific gene editing.

### BRIEF DESCRIPTIONS OF THE DRAWING

FIGS. 1A-1G show the sequencing and data analysis workflow. FIG. 1A: Barcodes were added to both forward and backward PCR primers and the pooled PCR products of multiple samples were sequenced with Oxford nanopore sequencing. FIG. 1B: De-multiplexing strategy of NanoBinner. NanoBinner aligns a read to barcode sequences with 256 bp amplicon sequence immediately next to it. The 256 bp amplicon sequence acts as an anchor so that the matching of barcodes is at the correct position. NanoBinner assigns a read to a barcode if the Phred scale mapping quality score was ≥30. FIG. 1C: Joint quantification of CAG and CCG repeat sizes using NanoRepeat. NanoRepeat first estimates the upper and lower bounds of the CAG and CCG repeat sizes separately, and then performs a joint quantification to refine the repeat sizes. In the joint quantification step, NanoRepeat generates a series of template sequences with m CAG repeats and n CCG repeats, where m and n are all integers within the upper and lower bounds determined in the first step. A read was aligned to this series of template sequences. The CAG and CCG repeat sizes that maximize the alignment score was the final estimates of the repeat sizes of the read. FIG. 1D: NanoRepeat separates reads using Gaussian mixture model (GMM). CAG and CCG repeat sizes are used as input features. The scatter plot shows the CAG and CCG repeat sizes of a typical example. The shading of the points indicates the number of reads. Model selection is performed to select the best number of Gaussian models. After filtering outliers, the two alleles are well separated (right). The dashed grey circles are equi-probability surfaces of the fitted Gaussian models where the probability outside the surface is less than 5%. FIG. 1E: The reads of each allele were error-corrected by Canu. SNPs were called from the error-corrected reads with longshot. The effects of on PAMs were examined. FIG. 1F: Locations of the PCR amplicons of each cohort. The lengths are the distance to the first nucleotide of the CAG repeat (based on GRCh38). FIG. 1G: Barcode design strategy used in this study. Top: The barcode design workflow. Bottom: The algorithm to find a set of barcodes where any two barcodes do not have any alignment. 1) Each barcode was a node in the graph. Initially, all nodes are connected in the undirected graph. 2) An all-vs-all alignment of the barcode sequences was performed, and the edge between two barcodes (nodes) was removed if the two barcodes were aligned. 3) The remaining edges only connect barcode pairs that have no alignment. Therefore, a complete subgraph (clique) is a set of barcodes in which any two barcodes have no alignment.

FIGS. 2A-2I provide an assessment of computational methods. FIGS. 2A-2C: Benchmarking repeat quantification on 2370 STR regions in the CHM13 genome. The average absolute error (avg_abs_error) of each method is shown. FIG. 2D: Scatter plot showing the CAG repeat size quantified by NanoRepeat and PCR-based fragment analysis. FIGS. 2E-2F: Repeat size difference between NanoRepeat's results and the PCR-based fragment analysis. FIG. 2G: Distribution of the fraction of reads of the longer allele. FIGS. 2H-2I: Histogram showing percentage of identical bases between the barcode sequence and the best aligned sequence in the human reference genome GRCh38. Sequences of 96 Oxford Nanopore barcodes were obtained from the online documentation of the PCR Barcoding Expansion Pack.

FIGS. 3A-3F show the CAG and CCG repeats detected from HD samples. FIG. 3A: Distribution of CAG repeat size of the French cohort. FIG. 3B: The normal and mHTT allele showed distinct distribution in CCG repeat sizes. FIG. 3C: CAG repeat size distribution of a typical HD sample. The reads derived from the normal allele (left) and the mHTT allele (right) are well separated. The estimated repeat sizes are marked by vertical grey lines. FIG. 3D: Scatter plot of a sample with a disease-causing allele (CAG repeat size=43) and an intermediate allele (CAG repeat size=34). The dashed grey circle is an equi-probability surface of the fitted Gaussian models where the probability outside the surface is less than 5%. FIG. 3E: Distribution of CAG repeat size of the same sample shown in FIG. 3D. Reads that are not confidently classified are removed. The two alleles are well separated (left, normal allele; right, mHTT allele). The estimated repeat sizes are marked by vertical lines. FIG. 3F: Histogram of the CAG repeat size of the CHDI cohort. The repeat was quantified by AmpRepeat using the Oxford Nanopore long-read sequencing data.

FIGS. 4A-4E show SNPs detected from the HD samples. FIG. 4A: Positions of 19 common SNPs (relative to the first base of the HTT exon-1). The box indicates the position of exon-1. FIG. 4B: Allele frequencies (AFs) of 19 common SNPs identified in the HD cohort. The details of the SNPs are shown in FIG. 4E. FIG. 4C: Scatter plot showing the of AFs of SNPs in HD mHTT alleles and the gnomAD database (NFE population). FIG. 4D: Scatter plot showing the of AFs of SNPs in normal alleles and the gnomAD database (NFE population). FIGS. 4B-4D are based on the data of the French HD cohort. FIG. 4E shows common SNPs identified in the HD cohort. Allele frequencies and effect on PAM are listed.

FIGS. 5A-5F show allele-specific cleavage mediated by SNP1 and SNP16. FIG. 5A: Cartoon depicting the relative position of SNP1 and SNP16-dependent PAMs flanking HTT exon-1 and one common PAM within HTT intron-1 (HDi3). PCR primer positions and estimated sizes of the targeted deleted sequences are indicated. 1, i3 and 16 are sgRNAs targeting PAMs at SNP1, HDi3 and SNP16, respectively. FIG. 5B: A genomic PCR showing HTT exon-1-targeted deletion mediated by SNP1 and SNP16-dependent PAMs. FIG. 5C: RT-qPCR analysis of HTT mRNA levels in HEK293 cells transfected with CRISPR enzymes targeting SNP1 and SNP16 (n=8). FIG. 5D: The haplotype of the ND33392 fibroblast cell line and the corresponding PAMs. FIGS. 5E-5F: A semiquantitative PCR reaction showing the reduction of the mHTT allele in a fibroblast cell line (ND33392) transfected with SaCas9 targeting SNP1 and SNP16. FIGS. 5C, 5F: The results are mean±SEM relative to the control group. (*p<0.05, one way ANOVA followed by Bonferroni's post-hoc).

FIGS. 6A-6G show haplotype analysis of HD individuals. FIG. 6A: Frequencies of top 10 haplotypes of HD samples. FIG. 6B: Violin plot showing the distribution of repeat sizes in different haplotypes of the mHTT chromosomes. The dots indicate the median of the repeat size. FIG. 6C: Scatter plot showing haplotype frequencies of the normal chromosomes and 1000 Genomes individuals (phase 3, non-Finnish European population). FIG. 6D: Scatter plot showing haplotype frequencies of the mHTT chromosomes and 1000 Genomes individuals (phase 3, non-Finnish European population). FIGS. 6E-6F: Frequencies of Hap1 in mHTT chromosomes in HD samples from different continents and ethnicities. FIG. 6G: Percentage of HD individuals who can be edited, based on the haplotype analysis.

### DETAILED DESCRIPTION OF THE INVENTION

Huntington's disease (HD) is an autosomal dominant neurodegenerative disease caused by CAG trinucleotide repeat expansions in exon-1 of huntingtin (HTT). Currently, there is no cure for HD, and the clinical care of individuals with HD is focused on symptom management. Allele-specific deletion of the expanded HTT allele (mHTT) can be accomplished by CRISPR/Cas9 by targeting nearby (<10 kb) SNPs that create or eliminate a Protospacer Adjacent Motif (PAM) near exon-1. Here, all potential PAM sites within a 10.4 kb genomic region flanking exon-1 of HTT was comprehensively analyzed in 983 individuals with HD using a multiplex targeted long-read sequencing approach on the Oxford Nanopore platform. Computational tools (NanoBinner and NanoRepeat) were developed to de-multiplex the data, detect repeats, and phase the reads on the expanded or the wild-type HTT allele. One SNP common to 30% of individuals with HD of European ancestry emerged through this analysis, which was confirmed as a strong candidate for allele-specific deletion of the mHTT in human HD cell lines. Additionally, up to 57% HD individuals may be candidates for allele-specific editing through combinatorial SNPs targeting. Cumulatively, a haplotype map is provided of the region surrounding exon-1 of HTT in individuals affected with HD. The workflow can be applied to other repeat expansion diseases to facilitate the design of guide RNAs for allele-specific gene editing.

The CRISPR/Cas9 system (Jinek, et al. (2012) Science 337:816-821; Cong, et al. (2013) Science 339:819-823; Mali, et al. (2013) Science 339:823-826) is a promising genome editing technology for genetic disorders such as HD. Indeed, a clinical trial using the CRISPR/Cas9 system for excising large portions of the HIV-1 genome in infected patients is currently ongoing (EBT-101; ClinicalTrials.gov Identifier: NCT05144386). In the CRISPR/Cas9 system, the Cas9 protein is coexpressed with guide RNAs (sgRNAs) that together form a ribonucleoprotein complex (sgRNA/Cas9 complex) which binds specific genomic DNA sequence and mediates a double-strand DNA (dsDNA) break. Targeted gene deletions can be made by non-homologous end joining (NHEJ) when a pair of sgRNA/Cas9 complexes bind either end of the DNA target and produce dsDNA breaks. Targeting specificity of the sgRNA/Cas9 complex is regulated by two factors: 1) the binding affinity of the 20-nt sgRNA with the complementary genomic DNA sequence; 2) the recognition of a protospacer adjacent motif (PAM) immediately following the genomic DNA/sgRNA complementary region (Jinek, et al. (2012) Science 337:816-821; Cong, et al. (2013) Science 339:819-823). While mismatches on the sgRNA/DNA complementary sequence are tolerable, the presence of an intact PAM motif is critical, and mutations on the PAM sequence cause ablation of cleavage (Anders, et al. (2014) Nature 513:569-573; Hsu, et al. (2013) Nat. Biotechnol., 31:827-832). Therefore, allele-specific gene editing can be achieved by taking advantage of SNPs that either eliminate or create PAM sequences.

Allele-specific editing of the mHTT allele in vitro and in vivo has been accomplished taking advantage of SNPs identified for use with the CRISPR/SpCas9 system (Monteys, et al. (2017) Mol. Ther., 25:12-23; Eisenstein, M. (2018) Nature 557: S42-S43; Shin, et al. (2016) Hum. Mol. Genet., 25:4566-4576). However, these studies mainly screened common SNPs in the normal population and a detailed haplotype map surrounding exon-1 of the HD population is unknown. To more broadly adapt this approach to the HD population, a robust, high throughput pipeline was developed to detect and phase all highly prevalent SNPs in the HD population that, when present, create a PAM cis to the expanded HTT allele that can be used together with the CRISPR systems. Notably, haplotype analysis of HD cohorts with a focus on allele specific ASO or miRNA-based silencing strategies targeting the spliced and unspliced transcript are available (Kay, et al. (2019) Am. J. Hum. Genet., 105:1112-1125; Warby, et al. (2009) Am. J. Hum. Genet., 84:351-366). Given that the HTT gene is 170 kb in length, most SNPs analyzed were distant from exon-1, yet for deletion of the CAG-repeat, a haplotype map surrounding exon-1 is required. Because editing efficacy reduces as with increasing distance from exon-1 (Monteys, et al. (2017) Mol. Ther., 25:12-23), the analysis was focused within 10 kb of exon-1.

To address this problem, Oxford Nanopore long-read sequencing paired with two novel computational tools (NanoBinner and NanoRepeat) was used to de-multiplex data, detect repeat size, and haplotype the allele. This was applied to 319 samples from the French HD consortium and validated on 664 samples from the CHDI Foundation, which consists of DNA samples from multiple continents and ethnic groups. All common SNPs in the French and CHDI HD cohorts were identified and all SNPs that provide PAMs to mediate CRISPR editing of the expanded HTT allele were analyzed.

Herein, therapeutics tools to inhibit mutant protein expression using genome-editing strategies based on the CRISPR/Cas9 technology are provided. Targeted gene deletions can be introduced when guide RNAs (e.g., two guide RNAs) complex with Cas9 and mediate dsDNA breaks followed by DNA repair. Given the potency of the CRISPR/Cas9 technology for targeting both alleles, and the fact that huntingtin is an important protein for cell viability, a concern of this method is that one will eliminate all huntingtin in the cell-good and bad. Thus, approaches that selectively target the expression of the mutant huntingtin allele are desirable. Here, a gene editing approach for specific targeting of the mutant huntingtin allele in the HD population is provided. The strategy takes advantage of single nucleotide polymorphisms prevalent in the HD population for which a targeting sequence (PAM motif) is generated depending on the nucleotide variation. gRNA sequences have been developed that recruit CRISPR/Cas9 complex to this allele-specific PAM motif. The safety and allele specificity may be examined in vitro using human cell lines (e.g., HEK293, NT2, HELA, neuronal precursor cell lines, HD fibroblasts (e.g., derived from human patients) and in vivo with a new transgenic mouse expressing the nucleotide variants within the HTT locus.

As explained above, the therapeutic benefit of RNA interference (RNAi) to reduce mutant huntingtin expression in different mouse models has been shown (Harper et al. (2005) Proc. Natl. Acad. Sci., 102:5820-5825; Boudreau et al. (2009) Mol. Ther., 17:1053-1063; Drouet et al. (2009) Annals of Neurol., 65:276-285). However, RNAi treatment does not completely eliminate mutant huntingtin expression, and the mutant protein remains present at low levels. This remaining protein may mitigate positive effects of RNAi therapy in HD patients. Thus, efforts to eliminate expression of all mutant HTT protein have been sought. Genome editing nucleases present such an opportunity.

Gene editing based on bacterial endonucleases such as CRISPR-associated protein-9 (Cas9) from Streptococcus pyogenes has revolutionized the field (Cong et al. (2013) Science 339:819-823; Ran et al. (2013) Nature Protocols 8:2281-2308; Mali et al. (2013) Science 339:823-826; Jinek et al. (2012) Science 337:816-821). The RNA-guided CRISPR/Cas9 system involves expressing Cas9 along with at least one guide RNA molecule (gRNA). When coexpressed, gRNAs bind and recruit Cas9 to a specific genomic target sequence where it mediates a double strand DNA (dsDNA) break and activates the dsDNA break repair machinery. Specific DNA fragments can be deleted when two gRNA/Cas9 complexes generate dsDNA breaks at relative proximity, and the genomic DNA (genDNA) is repaired by nonhomologous end joining. gRNAs were originally designed for recruiting the CRISPR/Cas9 complex to the HTT promoter to delete regulatory sequences and inhibit HTT expression. However, the complete elimination of the expression of both the normal and mutant alleles is likely not tolerable in human adults with HD. As such, an allele specific genome editing approach is provided herein where the CRISPR/Cas9 complex is targeted to the mutant HTT allele.

The binding specificity of the CRISPR/Cas9 complex depends on two different elements. First, the binding complementarity between the targeted genomic DNA sequence and the complementary recognition sequence of the gRNA. Second, the presence of a protospacer-adjacent motif (PAM) juxtaposed to the genomic DNA/gRNA complementary region. Whereas single point mutations in the complementary recognition sequence permit Cas9-mediated DNA cleavage, the preservation of an intact PAM motif is critical (Jinek et al. (2012) Science 337:816-821; Hsu et al. (2013) Nat. Biotech., 31:827-832; Sternberg et al. (2014) Nature 507:62-67). The PAM motif for S. pyogenes Cas9 has been fully characterized, and is NGG or NAG (Jinek et al. (2012) Science 337:816-821; Hsu et al. (2013) Nat. Biotech., 31:827-832). While any nucleotide type can be found at the first position of the PAM motif, a C/T nucleotide at position 2 and/or a C/T/A nucleotide at position 3 can disrupt the PAM motif and subsequently inhibit Cas9-mediated dsDNA cleavage. Thus, PAM motifs containing single nucleotide polymorphisms (SNP) at positions two or three will confer allele cleavage selectivity when targeted with CRISPR/Cas9 complexes.

The CAG (encoding glutamine) disease expansion in HTT is located within the 1^{st} exon of the HTT gene (The Huntington's Disease Collaborative Research Group (1993) Cell 72:971-983). A short exon 1 HTT polyadenylated mRNA resulting from aberrant splicing of the mutant allele is reported to be translated into a pathogenic exon 1 HTT protein and contribute to disease progression (Sathasivam et al. (2013) Proc. Natl. Acad. Sci., 110:2366-2370; Gipson et al. (2013) RNA Biol., 10:1647-1652). The main regulatory regions of the HTT promoter are located within the first 2 Kb upstream of the transcription start site (Holzmann et al. (2001) Brain Res., 92:85-97). The 1000 Genome database has reported the location and allele frequency of prevalent SNPs located in the promoter and within the first intronic sequence of the HTT gene (Abecasis et al. (2010) Nature 467:1061-1073).

Clustered, regularly interspaced, short palindromic repeat (CRISPR)/Cas9 technology is well known in the art (see, e.g., Sander et al. (2014) Nature Biotech., 32:347-355; Jinek et al. (2012) Science, 337:816-821). Cas9 possesses two nuclease domains, a RuvC-like nuclease domain and a HNH-like nuclease domain, and is responsible for the destruction of the target DNA (Jinek et al. (2012) Science, 337:816-821; Sapranauskas et al. (2011) Nucleic Acids Res. 39:9275-9282). The two nucleases generate double-stranded breaks. The double-stranded endonuclease activity of Cas9 requires a target sequence (e.g., ~20 nucleotides) and a short conserved sequence (~2-5 nucleotides; e.g., 3 nucleotides) known as protospacer-associated motif (PAM), which follows immediately 3′- of the CRISPR RNA (crRNA) complementary sequence (Jinek et al. (2012) Science, 337:816-821; Nishimasu et al. (2014) Cell 156 (5): 935-49; Swarts et al. (2012) PLOS One, 7: e35888; Sternberg et al. (2014) Nature 507 (7490): 62-7). Guidelines and computer-assisted methods for generating gRNAs are available (see, e.g, CRISPR Design Tool (crispr.mit.edu/); Hsu et al. (2013) Nat. Biotechnol. 31:827-832; www.addgene.org/CRISPR; and CRISPR gRNA Design tool-DNA2.0 (www.dna20.com/eCommerce/startCas9)). Typically, the PAM sequence is present in the DNA target sequence but not in the gRNA sequence.

As stated above, wild-type Cas9 creates a site-specific double-stranded DNA break. The double strand break can be repaired by non-homologous end joining (NHEJ) pathway yielding an insertion and/or deletion or, in the presence of a donor template, by homology-directed repair (HDR) pathway for replacement mutations (Overballe-Petersen et al. (2013) Proc. Natl. Acad. Sci. U.S.A. 110:19860-19865; Gong et al. (2005) Nat. Struct. Mol. Biol. 12:304-312). A Cas9 mutant may also be used in the instant invention (e.g., a mutant with an inactivated HNH and/or RuvC nuclease). In a particular embodiment, the mutant is Cas9 D10A. Cas9 D10A nicks single-strand DNA rather than generate a double strand break (Cong et al. (2013) Science, 339:819-823; Davis et al. (2014) Proc. Natl. Acad. Sci., 111: E924-932). The nicks are repaired by HDR pathway. Two (or more) gRNAs can be used to generate a staggered double strand break with Cas9 D10A.

In accordance with the instant invention, methods of treating, inhibiting, and/or preventing a polyglutamine disorder (e.g., Huntington's disease) are provided. In accordance with another aspect of the instant invention, methods for reducing the expression of a mutant protein (e.g., mutant huntingtin) encoded by an allele of a gene associated with a polyglutamine disorder (e.g., Huntington's disease) in a cell are provided. Polyglutamine (polyQ) disorder are generally neurodegenerative disorders which are caused by expanded cytosine-adenine-guanine (CAG) repeats (e.g., greater than about 36 repeats) encoding a long polyQ tract in the respective proteins. Polyglutamine (polyQ) disorders include, without limitation, spinocerebellar ataxia (SCA; types 1, 2, 3, 6, 7, 17), Machado-Joseph disease (MJD/SCA3), Huntington's disease (HD), dentatorubral pallidoluysian atrophy (DRPLA), and spinal and bulbar muscular atrophy, X-linked 1 (SMAX1/SBMA). In a particular embodiment, the methods of the instant invention comprise inhibiting, reducing, or eliminating mutant protein (e.g., HTT) expression. In a particular embodiment, the method comprises inactivating (e.g., cleaving and/or deleting (at least partially (e.g., deleting the first exon and, optionally, the promoter))) mutant alleles (e.g., HTT alleles) using CRISPR/Cas9 technology. In a particular embodiment, the method comprises administering at least one Cas9 (e.g., a nucleic acid molecule encoding Cas9) and at least one gRNA (e.g., a nucleic acid molecule encoding said gRNA) to said subject. Examples of Cas9 include, without limitation, Streptococcus pyogenes Cas9, Cas9 D10A, high fidelity Cas9 (Kleinstiver et al. (2016) Nature, 529:490-495; Slaymaker et al. (2016) Science, 351:84-88), Cas9 nickase (Ran et al. (2013) Cell, 154:1380-1389), Streptococcus pyogenes Cas9 with altered PAM specificities (e.g., SpCas9_VQR, SpCas9_EQR, and SpCas9_VRER; Kleinstiver et al. (2015) Nature, 523:481-485), Staphylococcus aureus Cas9, the CRISPR/Cpf1 system of Acidaminococcus, and the CRISPR/Cpf1 system of Lachnospiraceae. In a particular embodiment, the Cas9 is S. aureus Cas9. In a particular embodiment, the Cas9 is S. pyogenes Cas9. In a particular embodiment, the Cas9 has an inactivated HNH and/or RuvC nuclease, particularly Cas9 D10A. In a particular embodiment, at least two gRNAs are delivered. In a particular embodiment, more than two gRNAs are delivered. In a particular embodiment, at least one gRNA targets a region adjacent to a PAM present in only one allele (e.g., in the mutant allele). In a particular embodiment, at least two gRNA target a region adjacent to a PAM present in only one allele (e.g., in the mutant allele). In a particular embodiment, at least one (or at least two) targeted PAM is in the 5′UTR, promoter, or first intron. In a particular embodiment, gRNA which is not targeted to an allele specific PAM may be included. In a particular embodiment, at least one gRNA targets anywhere from the 5′UTR to the 3′UTR of the gene (e.g., HTT gene), particularly within the first intron. In a particular embodiment, the method further comprises the administration of a donor nucleic acid molecule (e.g., DNA; e.g., a nucleic acid molecule encoding the desired sequence). The donor DNA may be a replacement sequence (e.g., wild-type) for the sequence excised from the mutant. The nucleic acids of the instant invention may be administered consecutively (before or after) and/or at the same time (concurrently). The nucleic acid molecules may be administered in the same composition or in separate compositions. In a particular embodiment, the nucleic acid molecules are delivered in a single vector (e.g., a viral vector).

The methods of the instant invention may also comprise the administration of an additional therapeutic for Huntington's disease or the related disorder. Other therapeutics include, without limitation: haloperidol, tetrabenazine, amantadine, huntingtin antisense, huntingtin siRNA, antidepressants, and antianxiety medications. The nucleic acids of the instant invention and the other therapeutics may be administered consecutively (before and/or after) and/or at the same time (concurrently). The other therapeutics may be administered in the same composition or in separate compositions as the nucleic acid molecules of the instant invention.

In a particular embodiment, the nucleic acid molecules of the instant invention are delivered (e.g., via infection, transfection, electroporation, etc.) and expressed in cells via a vector (e.g., a plasmid), particularly a viral vector. The expression vectors of the instant invention may employ a strong promoter, a constitutive promoter, and/or a regulated promoter. In a particular embodiment, the nucleic acid molecules are expressed transiently. Examples of promoters are well known in the art and include, but are not limited to, RNA polymerase II promoters, the T7 RNA polymerase promoter, and RNA polymerase III promoters (e.g., U6 and H1; see, e.g., Myslinski et al. (2001) Nucl. Acids Res., 29:2502-09). Examples of expression vectors for expressing the molecules of the invention include, without limitation, plasmids and viral vectors (e.g., adeno-associated viruses (AAVs), adenoviruses, retroviruses, and lentiviruses). In a particular embodiment, the nucleic acids of the instant invention are contained within an AAV (e.g., AAV9).

In a particular embodiment, the guide RNA of the instant invention may comprise separate nucleic acid molecules. For example, one RNA specifically hybridizes to a target sequence (crRNA) and another RNA (trans-activating crRNA (tracrRNA)) which specifically hybridizes with the crRNA. In a particular embodiment, the guide RNA is a single molecule (sgRNA) which comprises a sequence which specifically hybridizes with a target sequence (crRNA; complementary sequence) and a sequence recognized by Cas9 (e.g., a tracrRNA sequence; scaffold sequence). Examples of gRNA scaffold sequences are well known in the art (e.g., 5′-GUUUUAGAGC UAGAAAUAGC AAGUUAAAAU AAGGCUAGUC CGUUAUCAAC UUGAAAAAGU GGCACCGAGU CGGUGCUUUU (SEQ ID NO: 1)). As used herein, the term “specifically hybridizes” does not mean that the nucleic acid molecule needs to be 100% complementary to the target sequence. Rather, the sequence may be at least 80%, 85%, 90%, 95%, 97%, 99%, or 100% complementary to the target sequences. The greater the complementarity reduces the likelihood of undesired cleavage events at other sites of the genome. In a particular embodiment, the region of complementarity (e.g., between a guide RNA and a target sequence) is at least about 10, at least about 12, at least about 15, at least about 17, at least about 20, at least about 25, at least about 30, at least about 35, or more nucleotides. In a particular embodiment, the region of complementarity (e.g., between a guide RNA and a target sequence) is about 15 to about 25 nucleotides, about 15 to about 23 nucleotides, about 16 to about 23 nucleotides, about 17 to about 23 nucleotides, about 17 to about 21 nucleotides, about 19 to about 23 nucleotides or about 20 nucleotides.

In a particular embodiment, at least two guide RNAs are used, wherein at least one first guide RNA targets a region 5′ of exon 1 (e.g., 5′ untranslated region, promoter, and/or within the first 5 kb 5′ of the transcription start site) and at least one second guide RNA targets a region 3′ of exon 1 (e.g., intron 1). In a particular embodiment, at least one first guide RNA targets a SNP (e.g., a target sequence adjacent to a PAM associated (e.g., gain or loss) with a SNP). In a particular embodiment, at least one second guide RNA targets a SNP (e.g., a target sequence adjacent to a PAM associated (e.g., gain or loss) with a SNP). In a particular embodiment, at least one first guide RNA and at least one second guide RNA target a SNP (e.g., a target sequence adjacent to a PAM associated (e.g., gain or loss) with a SNP). In a particular embodiment, at least one first guide RNA targets SNP4, SNP7, SNP8, SNP5, SNP9, SNP6, SNP10, SNP1, SNP2, SNP12, or SNP3. In a particular embodiment, at least one second guide RNA targets SNP13, SNP14, SNP15, SNP16, SNP17, SNP18, or SNP19.

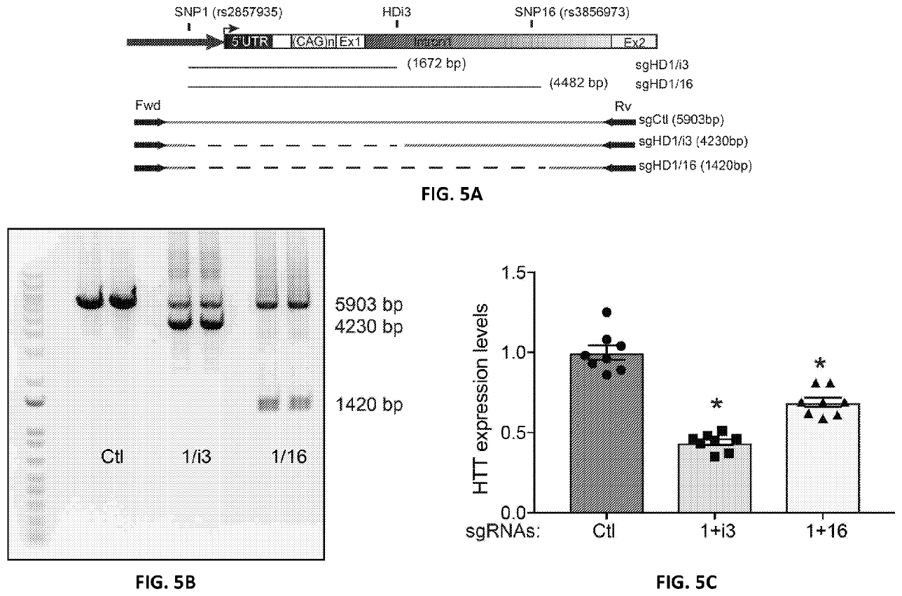

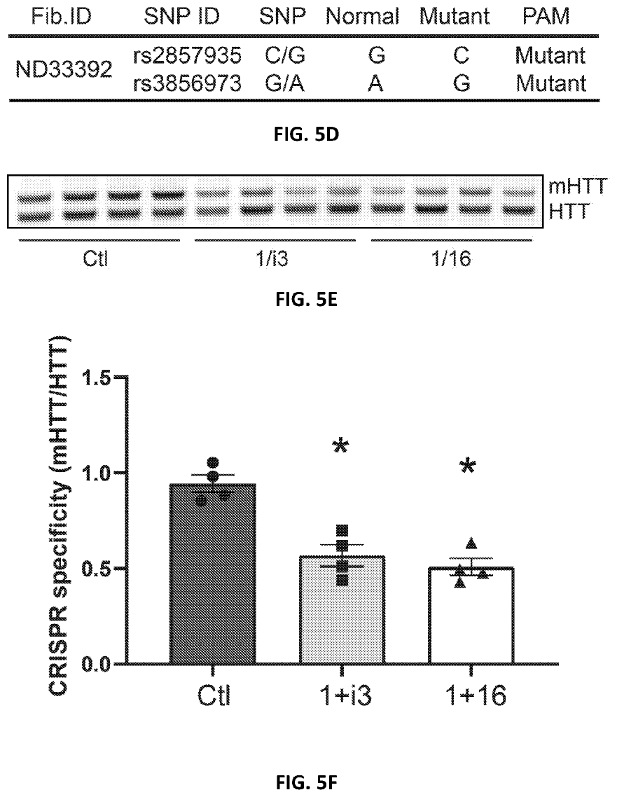

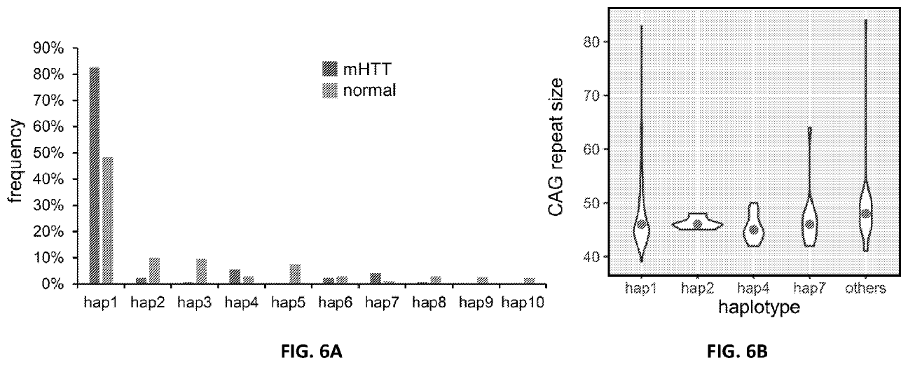

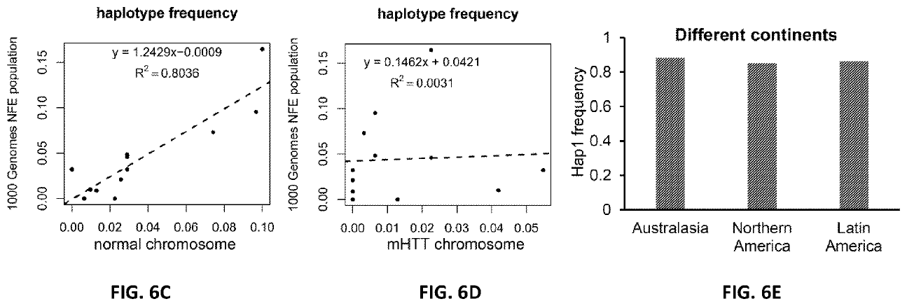

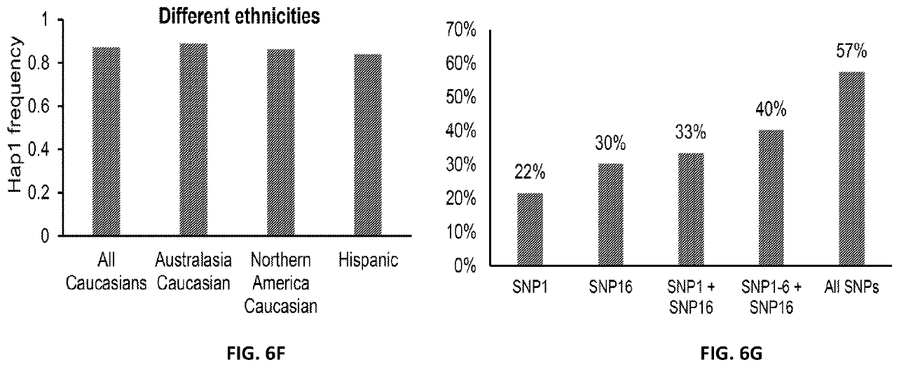

In a particular embodiment, at least one of the guide RNAs targets a sequence or comprises a sequence (inclusive of RNA version of DNA molecules (e.g., U in place of T)) as set forth herein and/or in the Example or Figures provided in WO 2017/062983, incorporated herein by reference (see, e.g., the guide or target sequences provided in FIGS. 5 and 6). In a particular embodiment, the guide RNA targets a sequence or comprises a sequence which has at least 80%, 85%, 90%, 95%, 97%, 99%, or 100% homology or identity to a sequence set forth in the Examples and/or Figures (e.g., FIGS. 5 and 6) provided in WO 2017/062983. In a particular embodiment, the guide RNA targets a sequence selected from the group consisting of SEQ ID NOs: 156-228 and 379-388 in WO 2017/062983. In a particular embodiment, the guide RNA comprises a sequence selected from the group consisting of SEQ ID NOs: 156-228 and 379-388 (e.g., in RNA form) in WO 2017/062983. The sequences may be extended or shortened by 1, 2, 3, 4, or 5 nucleotides at the end of the sequence opposite from the PAM (i.e., the 5′ end). When the sequence is extended the added nucleotides should correspond to the HTT sequence. In a particular embodiment, at least one gRNA comprises sgHD1, sgHD2, sgHD3, sgHD4, sgHD5c, sgHD6c, or sgHD6g in WO 2017/062983. In a particular embodiment, at least one gRNA comprises sgHDi3 or sgHDi4 in WO 2017/062983. In a particular embodiment, at least one gRNA comprises sgHD1 in WO 2017/062983. In a particular embodiment, at least one gRNA comprises sgHDi3 in WO 2017/062983.

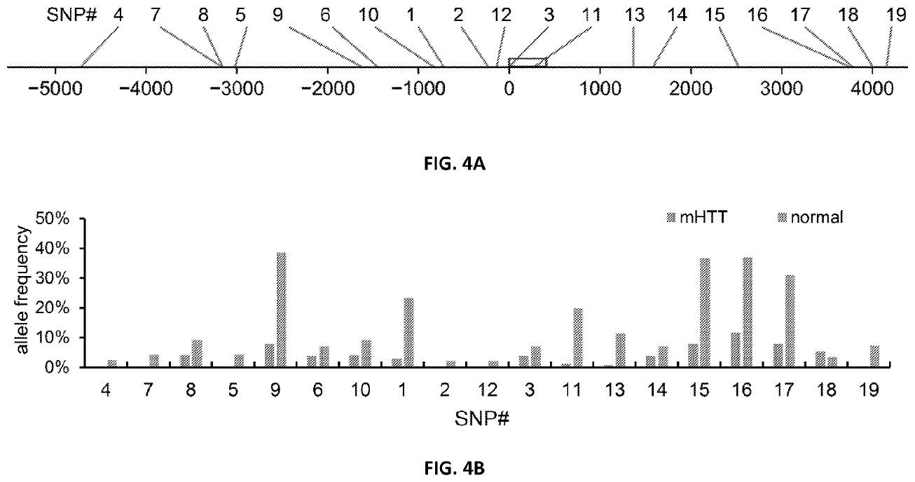

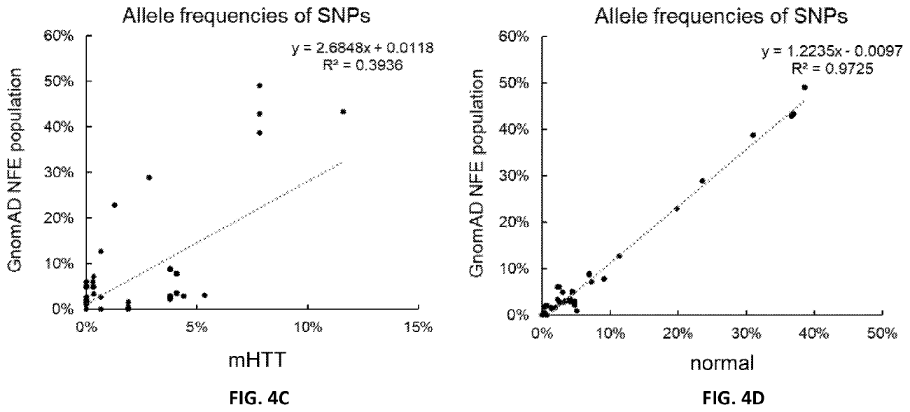

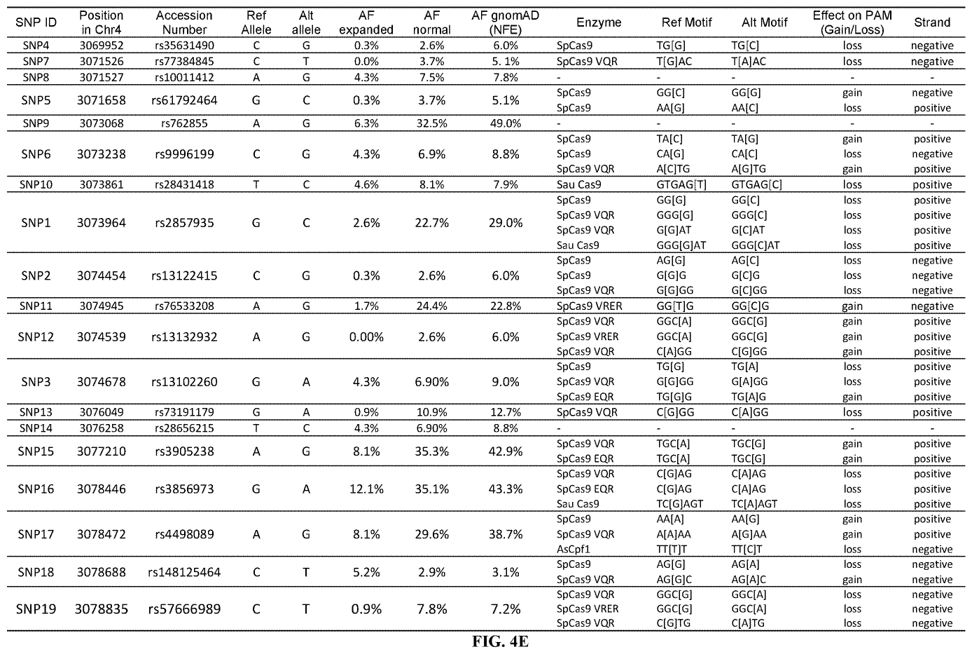

In a particular embodiment, at least one of the guide RNA targets a sequence or comprises a sequence (inclusive of RNA version of DNA molecules (e.g., U in place of T)) as set forth in the Example or Figures provided herein. In a particular embodiment, at least one of the guide RNA targets a sequence or comprises a sequence which has at least 80%, 85%, 90%, 95%, 97%, 99%, or 100% homology or identity to a sequence set forth in the Examples and/or Figures provided herein. The sequences may be extended or shortened by 1, 2, 3, 4, or 5 nucleotides at the end of the sequence opposite from the PAM (i.e., the 5′ end). When the sequence is extended, the added nucleotides should correspond to the HTT sequence. In a particular embodiment, at least one of the guide RNA targets a sequence of at one of the SNPs in FIG. 4E (e.g., at least one of SNP1-SNP16) or comprises a sequence of at one of the SNPs in FIG. 4E (e.g., at least one of SNP1-SNP16). For example, at least one of the guide RNA targets a sequence adjacent to the PAM associated with the SNP (e.g., SNP1-SNP16) or comprises a sequence adjacent to the PAM associated with the SNP (e.g., SNP1-SNP16).

Example sequences which can be included in or targeted by guide RNA include:

Sequence sgHD1 (SNP1)
Complementary guide:
(SEQ ID NO: 94)
5′GCTCCAGGCGTCGGCGG 3′
or
(SEQ ID NO: 95)
GCCCCGCTCCAGGCGTCGGCG
sgRNA expression cassette: hU6 promoter
Major Allele (C): Have a PAM motif on the Positive strand (NGG)
(SEQ ID NO: 96/SEQ ID NO: 97)
GTCGCCCCGCTCCAGGCGTCGGCGGGGGATCCTTTCCGCATGGGCCTGCGCC
CAGCGGGGCGAGGTCCGCAGCCGCCCCCTAGGAAAGGCGTACCCGGACGCGG
(SEQ ID NO: 94)
GCTCCAGGCGTCGGCGG
(SEQ ID NO: 139)
GCCCCGCTCCAGGCGTCGGCGG
Minor Allele (G): Disruption of the PAM motif
(SEQ ID NO: 98/SEQ ID NO: 99)
GTCGCCCCGCTCCAGGCGTCGGCGGGGCATCCTTTCCGCATGGGCCTGCGCC
CAGCGGGGCGAGGTCCGCAGCCGCCCCGTAGGAAAGGCGTACCCGGACGCGG
Sequence sgHD2 (SNP2)
Complementary guide:
(sgHD2; SEQ ID NO: 100)
5′GGCGCGGGGCTCAACGGAG 3′
sgRNA expression cassette: hU6 promoter
Major Allele (C): Has a PAM motif on the Negative strand (NGG)
(SEQ ID NO: 100)
GAGGCAACTCGGGGCGCGG
(SEQ ID NO: 101/SEQ ID NO: 102)
TTACAGTCTCACCACGCCCCGTCCCCTCTCCGTTGAGCCCCGCGCCTTC
AATGTCAGAGTGGTGCGGGGCAGGGGAGAGGCAACTCGGGGCGCGGAAG
Minor Allele (G): Disruption of the PAM motive
(SEQ ID NO: 103/SEQ ID NO: 104)
TTACAGTCTCACCACGCCCCGTCCGCTCTCCGTTGAGCCCCGCGCCTTC
AATGTCAGAGTGGTGCGGGGCAGGCGAGAGGCAACTCGGGGCGCGGAAG
Sequence sgHD3 (SNP3)
Complementary guide:
(sgHD3; SEQ ID NO: 105)
5′GTCTGGGACGCAAGGCGCCG3′
sgRNA expression cassette: hU6 promoter
Major Allele (G): Has a PAM motif on the Positive strand (NGG)
(SEQ ID NO 106/SEQ ID NO: 107)
GCGTCTGGGACGCAAGGCGCCGTGGGGGCTGCCGGGACGGGTCCAAGAT
CGCAGACCCTGCGTTCCGCGGCACCCCCGACGGCCCTGCCCAGGTTCTA
(SEQ ID NO: 105)
GTCTGGGACGCAAGGCGCCG
Minor Allele (A): Loss PAM motif on the Positive strand (NGG)
(SEQ ID NO: 108/SEQ ID NO: 109)
GCGTCTGGGACGCAAGGCGCCGTGAGGGCTGCCGGGACGGGTCCAAGAT
CGCAGACCCTGCGTTCCGCGGCACTCCCGACGGCCCTGCCCAGGTTCTA
Sequence sgHD4 (SNP4)
Complementary guide:
(SEQ ID NO: 110)
5′ GATGCACGCGGGGTGGGGC 3′
sgRNA expression cassette: hU6 promoter
Major Allele: Has a PAM motif on the negative strand (NGG))
(SEQ ID NO: 110)
3′ CGGGGGGGGCGCACGTAG 5′
(SEQ ID NO: 111/SEQ ID NO: 112)
5′ TCTGCGTCAGGGTTTCCTTCTTTTCCAGCCCCACCCCGCGTGCATCCCA 3′
3′ AGACGCAGTCCCAAAGGAAGAAAAGGTCGGGGGGGGCGCACGTAGGGT 5′
Minor Allele: SNP produces a Loss of the PAM motif.
(SEQ ID NO: 113/SEQ ID NO: 114)
5′ TCTGCGTCAGGGTTTCCTTCTTTTGCAGCCCCACCCCGCGTGCATCCCA 3′
3′ AGACGCAGTCCCAAAGGAAGAAAACGTCGGGGTGGGGCGCACGTAGGGT 5′
Sequence sgHD5g and sgHD5c (SNP5)
Complementary guide:
(sgHD5g; SEQ ID NO: 115)
5′ATTCAGGTTGATGTCCT 3′
Complementary guide:
(sgHD5c; SEQ ID NO: 116)
5′ATCCCATTCTGAGGTTCTGG 3
sgRNA expression cassette: hH1 promoter
Major Allele: Has a PAM motif on the Positive strand (NAG)
(SEQ ID NO: 117/SEQ ID NO: 118)
5′ CCCTCATTCAGGTTGATGTCCTAAGCCCCAGAACCTCAGAATGGGATTG 3′
3′ GGGAGTAAGTCCAACTACAGGATTCGGGGTCTTGGAGTCTTACCCTAAC 5′
(SEQ ID NO: 115)
ATTCAGGTTGATGTCCT
Minor Allele: generates a PAM motif on the Negative strand (NAG)
(SEQ ID NO: 116)
GGTCTTGGAGTCTTACCCTA
(SEQ ID NO: 119/SEQ ID NO: 120)
5′ CCCTCATTCAGGTTGATGTCCTAACCCCCAGAACCTCAGAATGGGATTG 3′
3′ GGGAGTAAGTCCAACTACAGGATTGGGGGTCTTGGAGTCTTACCCTAAC 5′
Sequence sgHD6c and sgHD6g (SNP6)
Complementary guide:
(sgHD6c; SEQ ID NO: 121)
5′GCAGGCAGAGAGGAGCC 3′
Complementary guide:
(sgHD6g; SEQ ID NO: 122)
5′GCCTGGCTAAAGTAGGCTT 3′
sgRNA expression cassette: hU6 promoter
Major Allele (C): Have a PAM motif on the negative strand (NAG)
(SEQ ID NO: 121)
CCGAGGAGAGACGGACG
(SEQ ID NO: 123/SEQ ID NO:124)
5′ GTGGCCTGGCTAAAGTAGGCTTTACTGGGCTCCTCTCTGCCTGCATCAC 3′
3′ CACCGGACCGATTTCATCCGAAATGACCCGAGGAGAGACGGACGTAGTG 5′
Minor Allele (G): Generates a PAM motif on the Positive strand (NAG)
(SEQ ID NO: 125/SEQ ID NO: 126)
5′ GTGGCCTGGCTAAAGTAGGCTTTAGTGGGCTCCTCTCTGCCTGCATCAC 3′
3′ CACCGGACCGATTTCATCCGAAATCACCCGAGGAGAGACGGACGTAGTG 5′
(SEQ ID NO: 122)
GCCTGGCTAAAGTAGGCTT
Sequence sgHDi3
Complementary guide:
(SEQ ID NO: 127)
5′GCTTTTAGGACGCCTCGG 3′
or
(SEQ ID NO: 128)
GTGCTTTTAGGACGCCTCGGC
sgRNA expression cassette: hU6 promoter
(SEQ ID NO: 129/SEQ ID NO: 130)
5′ AATGCTTTTAGGACGCCTCGGCGGGAGTGGCGGGGCAGGGGGGGGGCG 3
3′ TTACGAAAATCCTGCGGAGCCGCCCTCACCGCCCCGTCCCCCCCCCGC 5′
(SEQ ID NO: 127)
GCTTTTAGGACGCCTCGG
(SEQ ID NO: 128)
GTGCTTTTAGGACGCCTCGGC
Sequence sgHDi4
Complementary guide:
(sgHDi4; SEQ ID NO: 131)
5′ GCGGGACACTTCGAGAGG 3′
sgRNA expression cassette: hU6 promoter
(SEQ ID NO: 132/SEQ ID NO: 133)
5′ GGCGCGGGACACTTCGAGAGGAGGCGGGGTTTGGAGCTGGAGAGATGT 3′
3′ CCGCGCCCTGTGAAGCTCTCCTCCGCCCCAAACCTCGACCTCTCTACA 5′
(SEQ ID NO: 131)
GCGGGACACTTCGAGAGG
SNP16
Ref. Allele (G)
(SEQ ID NO: 134)
AATTGGGACTGTAAAGGGATAG
(SEQ ID NO: 135/SEQ ID NO: 136)
5′ TTAAAAATAAAAATAAGTTAACACTCGATTAACCCTGACATTTCCCTATCC 3′
3′ AATTTTTATTTTTATTCAATTGTGAGCTAATTGGGACTGTAAAGGGATAGG 5′
Minor Allele (A)
(SEQ ID NO: 137/SEQ ID NO: 138)
5′ TTAAAAATAAAAATAAGTTAACACTTGATTAACCCTGACATTTCCCTATCC 3′
3′ AATTTTTATTTTTATTCAATTGTGAACTAATTGGGACTGTAAAGGGATAGG 5′

In a particular embodiment, the methods of the instant invention comprise the use of at least two guide RNA. In a particular embodiment, the guide RNA targets a SNP of the instant invention (e.g., Table 6 of FIG. 4E). For example, the guide RNA may target a sequence adjacent to a PAM associated (e.g., gain or loss) with a SNP. In a particular embodiment, at least one of the guide RNA targets SNP16 (e.g., rs3856973). In a particular embodiment, at least one guide RNA comprises GATAGGGAAATGTCAGGGTTAA (SEQ ID NO: 134). In a particular embodiment, at least one of the guide RNA targets SNP1 (e.g., rs2857935). In a particular embodiment, at least one guide RNA comprises GCCCCGCTCCAGGCGTCGGCG (SEQ ID NO: 95). In a particular embodiment, at least one of the guide RNA targets SNP1 (e.g., rs2857935) and at least one of the guide RNA targets SNP16 (e.g., rs3856973). In a particular embodiment, the guide RNAs target SNP1, SNP2, SNP3, SNP4, SNP5, SNP6, and SNP16 (e.g., the method uses at least one guide RNA which targets each of the SNP). In a particular embodiment, the guide RNAs target all of the SNPs presented herein (e.g., Table 6 and FIG. 4E). In a particular embodiment, the guide RNAs target SNP1-SNP16. In a particular embodiment, the guide RNAs target SNP1-SNP10 and SNP12-SNP16.

As stated hereinabove, the instant invention provides nucleic acid molecules, vectors, and compositions and methods for the inhibition, treatment, and/or prevention of Huntington's disease and related disorders. Compositions comprising at least one nucleic acid described herein are also encompassed by the instant invention. In a particular embodiment, the composition comprises at least one, particularly at least two, guide RNA (e.g., a nucleic acid molecule encoding the guide RNA (e.g., an expression vector)) and at least one pharmaceutically acceptable carrier. The composition may further comprise at least one Cas9 (e.g., a nucleic acid molecule encoding Cas9) and/or at least one donor nucleic acid molecule. The composition may further comprise at least one additional therapeutic (as described above). In a particular embodiment, all of the nucleic acid molecules are encoded within a single expression vector (e.g., viral vector (e.g., AAV)). Alternatively, the other nucleic acid molecules may be contained within a separate composition(s) with at least one pharmaceutically acceptable carrier. The present invention also encompasses kits comprising a first composition comprising at least one guide RNA (e.g., a nucleic acid molecule encoding the guide RNA (e.g., an expression vector)) and a second composition comprising at least one Cas9 (e.g., a nucleic acid molecule encoding Cas9) and/or at least one donor nucleic acid molecule. The first and second compositions may further comprise at least one pharmaceutically acceptable carrier. In a particular embodiment, the kits of the instant invention comprise a first composition comprising at least one (or at least two) guide RNA (e.g., a nucleic acid molecule encoding the guide RNA (e.g., an expression vector)), at least one Cas9 (e.g., a nucleic acid molecule encoding Cas9), and/or at least one donor nucleic acid molecule (optionally all within a single vector) and a second composition comprising at least one additional therapeutic. The first and second compositions may further comprise at least one pharmaceutically acceptable carrier.

As explained hereinabove, the compositions of the instant invention are useful for treating Huntington's disease and related disorders. A therapeutically effective amount of the composition may be administered to a subject in need thereof. The dosages, methods, and times of administration are readily determinable by persons skilled in the art, given the teachings provided herein.

The components as described herein will generally be administered to a patient as a pharmaceutical preparation. The term “patient” or “subject” as used herein refers to human or animal subjects. The components of the instant invention may be employed therapeutically, under the guidance of a physician for the treatment of the indicated disease or disorder.

The pharmaceutical preparation comprising the components of the invention may be conveniently formulated for administration with an acceptable medium (e.g., pharmaceutically acceptable carrier) such as water, buffered saline, ethanol, polyol (for example, glycerol, propylene glycol, liquid polyethylene glycol and the like), dimethyl sulfoxide (DMSO), oils, detergents, suspending agents or suitable mixtures thereof. The concentration of the agents in the chosen medium may be varied and the medium may be chosen based on the desired route of administration of the pharmaceutical preparation. Except insofar as any conventional media or agent is incompatible with the agents to be administered, its use in the pharmaceutical preparation is contemplated.

Selection of a suitable pharmaceutical preparation depends upon the method of administration chosen. For example, the components of the invention may be administered by direct injection into any desired tissue (e.g., brain) or into the surrounding area. In this instance, a pharmaceutical preparation comprises the components dispersed in a medium that is compatible with blood or the target tissue.

The therapy may be, for example, administered parenterally, by injection into the blood stream (e.g., intravenous), or by subcutaneous, intramuscular or intraperitoneal injection. Pharmaceutical preparations for injection are known in the art. If injection is selected as a method for administering the therapy, steps must be taken to ensure that sufficient amounts of the molecules reach their target cells to exert a biological effect.

Pharmaceutical compositions containing a compound of the present invention as the active ingredient in intimate admixture with a pharmaceutical carrier can be prepared according to conventional pharmaceutical compounding techniques. The carrier may take a wide variety of forms depending on the form of preparation desired for administration, e.g., intravenous, oral or parenteral. In preparing the antibody in oral dosage form, any of the usual pharmaceutical media may be employed, such as, for example, water, glycols, oils, alcohols, flavoring agents, preservatives, coloring agents and the like in the case of oral liquid preparations (such as, for example, suspensions, elixirs and solutions); or carriers such as starches, sugars, diluents, granulating agents, lubricants, binders, disintegrating agents and the like in the case of oral solid preparations (such as, for example, powders, capsules and tablets). Injectable suspensions may be prepared, in which case appropriate liquid carriers, suspending agents and the like may be employed.

A pharmaceutical preparation of the invention may be formulated in dosage unit form for ease of administration and uniformity of dosage. Dosage unit form, as used herein, refers to a physically discrete unit of the pharmaceutical preparation appropriate for the patient undergoing treatment. Each dosage should contain a quantity of active ingredient calculated to produce the desired effect in association with the selected pharmaceutical carrier. Procedures for determining the appropriate dosage unit are well known to those skilled in the art. Dosage units may be proportionately increased or decreased based on the weight of the patient. Appropriate concentrations for alleviation of a particular pathological condition may be determined by dosage concentration curve calculations, as known in the art.

The methods of the instant invention may further comprise monitoring the disease or disorder in the subject after administration of the composition(s) of the instant invention to monitor the efficacy of the method. For example, the subject may be monitored for characteristics of Huntington's disease and related disorders.

The methods of the instant invention may further comprise determining if the patient has the SNP prior to treatment. For example, the genome of the patient may be sequenced to identify if the patient has a SNP targeted by the gRNA to be administered.

In accordance with another aspect of the instant invention, methods of identifying target sequences for gRNA are provided. In a particular embodiment, the methods provide for the identification of guide RNAs for allele-specific gene editing. In a particular embodiment, the method is for identifying guide RNAs for allele-specific gene editing of a gene associated with a disease or disorder, such as a repeat expansion disorder such as a polyglutamine disorder (e.g., Huntington's disease). In a particular embodiment, the method is for identifying guide RNAs for allele-specific gene editing to reduce and/or eliminate the expression of a mutant protein (e.g., mutant huntingtin) encoded by an allele of a gene associated with a disease or disorder, such as a repeat expansion such as a polyglutamine disorder (e.g., Huntington's disease). In certain embodiments, the method is computer-assisted. In certain embodiments, the method is as described in the Example. In certain embodiments, the method comprises PCR amplification of the target region (e.g., barcoded PCR), sequencing the PCR products, de-multiplexing the sequencing data (e.g., with NanoBinner), detecting disease-associated repeats (e.g., CAG repeats) optionally using NanoRepeat, and identifying SNPs within diseased alleles which affect the presence or absence of PAMs.

### Definitions

The singular forms “a,” “an,” and “the” include plural referents unless the context clearly dictates otherwise.

The terms “isolated” is not meant to exclude artificial or synthetic mixtures with other compounds or materials, or the presence of impurities that do not interfere with the fundamental activity, and that may be present, for example, due to incomplete purification, or the addition of stabilizers.

“Pharmaceutically acceptable” indicates approval by a regulatory agency of the Federal or a state government or listed in the U.S. Pharmacopeia or other generally recognized pharmacopeia for use in animals, and more particularly in humans.

A “carrier” refers to, for example, a diluent, preservative, antioxidant, solubilizer, emulsifier, adjuvant, excipient, bulking substances, auxiliary agent or vehicle with which an active agent of the present invention is administered. Pharmaceutically acceptable carriers can be sterile liquids, such as water and oils, including those of petroleum, animal, vegetable or synthetic origin, such as peanut oil, soybean oil, mineral oil, sesame oil and the like. Water or aqueous saline solutions and aqueous dextrose and glycerol solutions are preferably employed as carriers, particularly for injectable solutions. Suitable pharmaceutical carriers are described, for example, in “Remington's Pharmaceutical Sciences” by E. W. Martin.

The term “treat” as used herein refers to any type of treatment that imparts a benefit to a patient suffering from an injury, including improvement in the condition of the patient (e.g., in one or more symptoms), delay in the progression of the condition, etc.

As used herein, the term “prevent” refers to the prophylactic treatment of a subject who is at risk of developing a condition and/or sustaining an injury, resulting in a decrease in the probability that the subject will develop conditions associated with the injury.

A “therapeutically effective amount” of a compound or a pharmaceutical composition refers to an amount effective to prevent, inhibit, or treat a particular injury and/or the symptoms thereof. For example, “therapeutically effective amount” may refer to an amount sufficient to modulate the pathology associated traumatic brain injury in a patient.

As used herein, the term “subject” refers to an animal, particularly a mammal, particularly a human.

A “vector” is a genetic element, such as a plasmid, cosmid, bacmid, phage or virus, to which another genetic sequence or element (either DNA or RNA) may be attached so as to bring about the replication and/or expression of the attached sequence or element. A vector may be either RNA or DNA and may be single or double stranded. A vector may comprise expression operons or elements such as, without limitation, transcriptional and translational control sequences, such as promoters, enhancers, translational start signals, polyadenylation signals, terminators, and the like, and which facilitate the expression of a polynucleotide or a polypeptide coding sequence in a host cell or organism.

The following example describes illustrative methods of practicing the instant invention and is not intended to limit the scope of the invention in any way.

### Example

### Material and Methods

### HD Subjects

HD subjects from the French cohort were recruited in Paris at the Pitié Salpetrière Hospital in Paris, France. All subjects gave written informed consent, and blood samples were collected in accordance with local French regulations (Paris Necker ethics committee approval [RBM 03-48]). Genomic DNA samples from the CHDI cohort were generously provided by the participants in the Enroll-HD study and made available by CHDI Foundation, Inc. Enroll-HD is a global clinical research platform intended to accelerate progress towards therapeutics for Huntington's disease; certain samples and core datasets are collected annually on all research participants as part of this multi-center longitudinal observational study. Enroll-HD is sponsored by CHDI Foundation, Inc., a nonprofit biomedical research organization exclusively dedicated to developing therapeutics for Huntington's disease.

### Barcode Design for Multiplexed Long-Read Sequencing

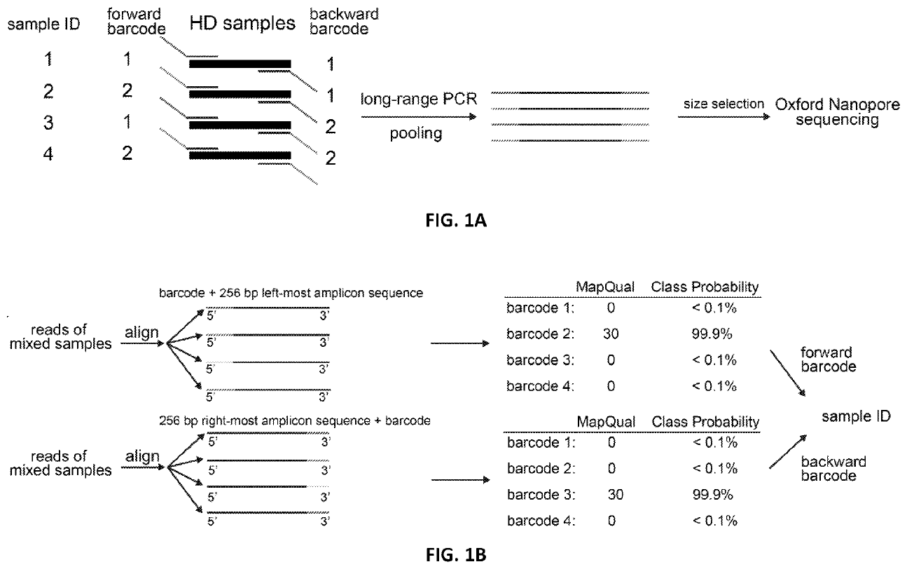

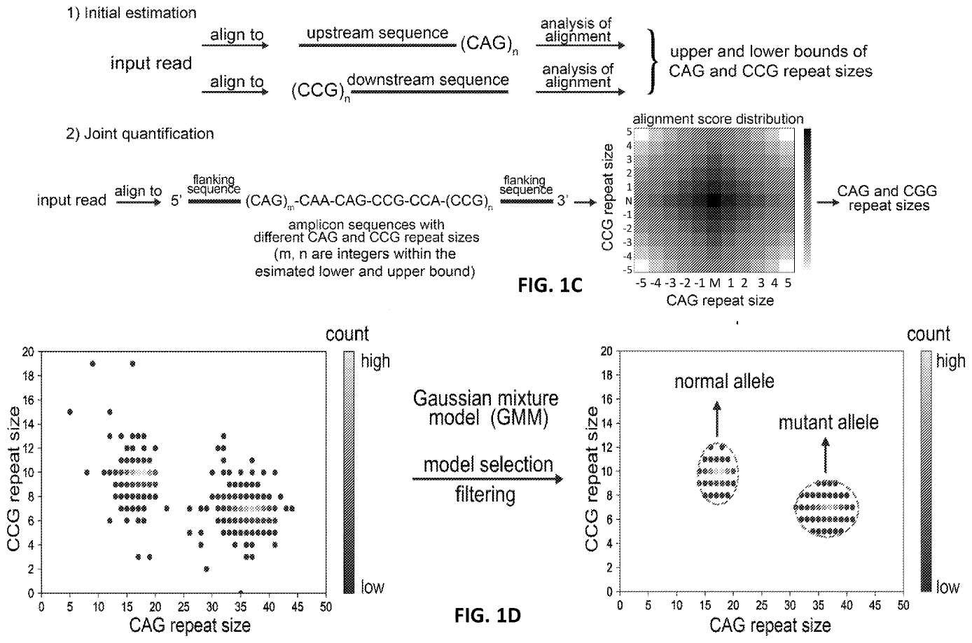

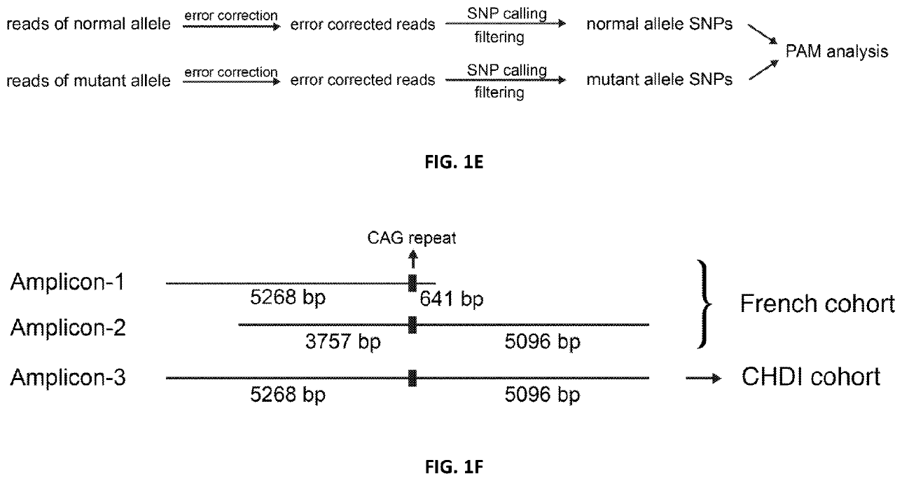

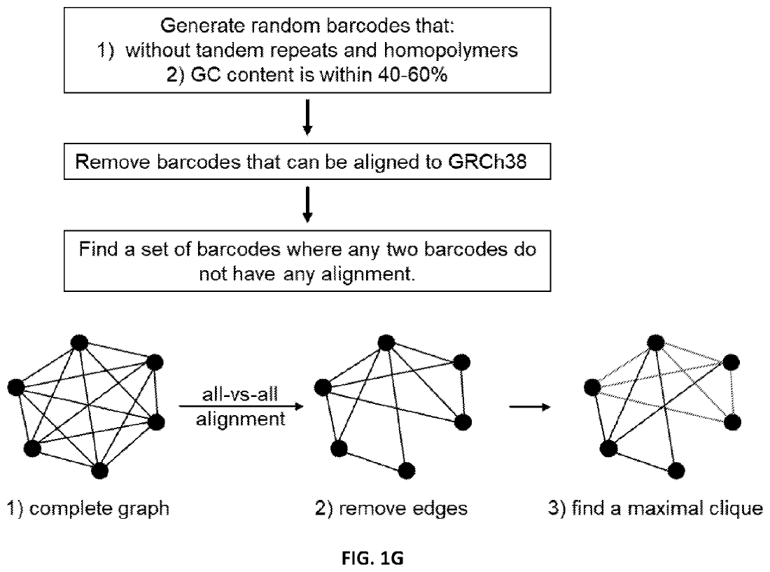

The barcode design workflow used in this study is shown in FIG. 1G. In-house barcode sequences were designed which met the following criteria: 1) there was no homopolymer that was longer than 2 bp (e.g, “TT” was allowed but “TTT” was not allowed); 2) there was no tandem repeat; 3) GC content is within 40-60%; 4) the barcode sequence cannot be mapped to human reference genome GRCh38 so that its binding with human genomic DNA during the PCR process is minimized; 5) pairwise sequence similarity between every two barcodes was minimized. To meet this goal, an excessive amount (20,000) of barcode sequences was generated that met criteria 1-3. Next, the barcode sequences were aligned to the GRCh38 reference genome using the blastn algorithm (Altschul, et al. (1990) J. Mol. Biol., 215:403-410; Camacho, et al. (2009) BMC Bioinformatics 10:421) and any barcodes aligned to GRCh38 were removed. Then a graph-based algorithm was used to find a set of barcodes where there was no pairwise alignment (blastn algorithm, word size=6) between any two barcodes (FIG. 1G). Each barcode was a node in the graph. Initially, all nodes are connected in the undirected graph. An all-vs-all alignment of the barcode sequences was performed using the blastn algorithm (word size=6). The edge between two barcodes (nodes) was removed if the two barcodes were aligned. The remaining edges only connect barcode pairs that have no alignment. Therefore, a complete subgraph (clique) is a set of barcodes in which any two barcodes have no alignment. The network python package was used to find the cliques with enough nodes (number of barcodes). A set of 16-bp barcodes was designed for amplicon-1 (Table 2, FIG. 1G). After analysis of the sequencing data of Amplicon-1, it was found that the first few bases were trimmed in some of the reads and thus longer barcode sequences might be better for the de-multiplexing process. 32-bp barcodes were designed for amplicon-2 (Table 3) and amplicon-3 (Table 4).

TABLE 2

| primer ID | primer sequence | SEQ ID NO: |
|---|---|---|
| A1B01F | 5′-AATTCGCCAGTGATGC-AAAACGAGGGTTGTCAAAGACCCCA-3 ′ | 2 |
| A1B02F | 5′-CAGCCATTGATGTCGA-AAAACGAGGGTTGTCAAAGACCCCA-3 ′ | 3 |
| A1B03F | 5′-GGCCGCTAGTAATTCA-AAAACGAGGGTTGTCAAAGACCCCA-3 ′ | 4 |
| A1B04F | 5′-TCAGGCGCCGATTAAT-AAAACGAGGGTTGTCAAAGACCCCA-3 ′ | 5 |
| A1B05F | 5′-ACTAAGCGAGGTCTCT-AAAACGAGGGTTGTCAAAGACCCCA-3 ′ | 6 |
| A1B06F | 5′-CGTTCCATCGAGTAAG-AAAACGAGGGTTGTCAAAGACCCCA-3 ′ | 7 |
| A1B07F | 5′-ACTGTTCAGACGATCG-AAAACGAGGGTTGTCAAAGACCCCA-3 ′ | 8 |
| A1B08F | 5′-CCTCGACGTGGATAAT-AAAACGAGGGTTGTCAAAGACCCCA-3 ′ | 9 |
| A1B09F | 5′-GTACATCGGATGATCC-AAAACGAGGGTTGTCAAAGACCCCA-3 ′ | 10 |
| A1B10F | 5′-TACGGCGCTATTGAAC-AAAACGAGGGTTGTCAAAGACCCCA-3 ′ | 11 |
| A1B01R | 5′-AATTCGCCAGTGATGC-GAGGGAAGTGGCACTGAGCAAATCT-3′ | 12 |
| A1B02R | 5′-CAGCCATTGATGTCGA-GAGGGAAGTGGCACTGAGCAAATCT-3′ | 13 |
| A1B03R | 5′-GGCCGCTAGTAATTCA-GAGGGAAGTGGCACTGAGCAAATCT-3′ | 14 |
| A1B04R | 5′-TCAGGCGCCGATTAAT-GAGGGAAGTGGCACTGAGCAAATCT-3′ | 15 |
| A1B05R | 5′-ACTAAGCGAGGTCTCT-GAGGGAAGTGGCACTGAGCAAATCT-3′ | 16 |
| A1B06R | 5′-CGTTCCATCGAGTAAG-GAGGGAAGTGGCACTGAGCAAATCT-3′ | 17 |
| A1B07R | 5′-ACTGTTCAGACGATCG-GAGGGAAGTGGCACTGAGCAAATCT-3′ | 18 |
| A1B08R | 5′-CCTCGACGTGGATAAT-GAGGGAAGTGGCACTGAGCAAATCT-3′ | 19 |
| A1B09R | 5′-GTACATCGGATGATCC-GAGGGAAGTGGCACTGAGCAAATCT-3 ′ | 20 |
| A1B10R | 5′-TACGGCGCTATTGAAC-GAGGGAAGTGGCACTGAGCAAATCT-3′ | 21 |

Barcoded primers for amplicon-1. The left part in the sequence is the barcode.
F: forward; R: reverse.

TABLE 3

| | | SEQ |
|---|---|---|
| primer | | ID |
| ID | primer sequence | NO: |
| A2B01F | 5′-CGTCGTTAACAGCGTACAGCCATTGATGTCGA- | 22 |
| | AAAGTCCCGATGATCCATTGCCTCC-3′ | |
| A2B02F | 5′-CGATAGTCTTACGAGCGGCCGCTAGTAATTCA- | 23 |
| | AAAGTCCCGATGATCCATTGCCTCC-3′ | |
| A2B03F | 5′-GCGAACGATCAGTCTTTCAGGCGCCGATTAAT- | 24 |
| | AAAGTCCCGATGATCCATTGCCTCC-3′ | |
| A2B04F | 5′-TGCCATGGCGTATACAACTAAGCGAGGTCTCT- | 25 |
| | AAAGTCCCGATGATCCATTGCCTCC-3′ | |
| A2B05F | 5′-AGCGCATCATTGGCATCGTTCCATCGAGTAAG- | 26 |
| | AAAGTCCCGATGATCCATTGCCTCC-3′ | |
| A2B06F | 5′-GACGACGTATGTACCTAATTCGCCAGTGATGC- | 27 |
| | AAAGTCCCGATGATCCATTGCCTCC-3′ | |
| A2B07F | 5′-ATAAGTTGCGCACGCTACTGTTCAGACGATCG- | 28 |
| | AAAGTCCCGATGATCCATTGCCTCC-3′ | |
| A2B08F | 5′-ATAACACGGTCCGGTTCCTCGACGTGGATAAT- | 29 |
| | AAAGTCCCGATGATCCATTGCCTCC-3′ | |
| A2B09F | 5′-GGTTAGATTCACGACCGTACATCGGATGATCC- | 30 |
| | AAAGTCCCGATGATCCATTGCCTCC-3′ | |
| A2B10F | 5′-AACGGTTCATGAGCCTTACGGCGCTATTGAAC- | 31 |
| | AAAGTCCCGATGATCCATTGCCTCC-3′ | |
| A2B01R | 5′-CGTCGTTAACAGCGTACAGCCATTGATGTCGA- | 32 |
| | ACAAACCTGATAACGCAAGCTACTGC-3′ | |
| A2B02R | 5′-CGATAGTCTTACGAGCGGCCGCTAGTAATTCA- | 33 |
| | ACAAACCTGATAACGCAAGCTACTGC-3′ | |
| A2B03R | 5′-GCGAACGATCAGTCTTTCAGGCGCCGATTAAT- | 34 |
| | ACAAACCTGATAACGCAAGCTACTGC-3′ | |
| A2B04R | 5′-TGCCATGGCGTATACAACTAAGCGAGGTCTCT- | 35 |
| | ACAAACCTGATAACGCAAGCTACTGC-3′ | |
| A2B05R | 5′-AGCGCATCATTGGCATCGTTCCATCGAGTAAG- | 36 |
| | ACAAACCTGATAACGCAAGCTACTGC-3′ | |
| A2B06R | 5′-GACGACGTATGTACCTAATTCGCCAGTGATGC- | 37 |
| | ACAAACCTGATAACGCAAGCTACTGC-3′ | |
| A2B07R | 5′-ATAAGTTGCGCACGCTACTGTTCAGACGATCG- | 38 |
| | ACAAACCTGATAACGCAAGCTACTGC-3′ | |
| A2B08R | 5′-ATAACACGGTCCGGTTCCTCGACGTGGATAAT- | 39 |
| | ACAAACCTGATAACGCAAGCTACTGC-3′ | |
| A2B09R | 5′-AACGGTTCATGAGCCTTACGGCGCTATTGAAC- | 40 |
| | ACAAACCTGATAACGCAAGCTACTGC-3′ | |
| A2B10R | 5′-GGTTAGATTCACGACCGTACATCGGATGATCC- | 41 |
| | ACAAACCTGATAACGCAAGCTACTGC-3′ | |

Barcoded primers for amplicon-2. The left part in the
sequence is the barcode. F: forward; R: reverse.

TABLE 4

| | | SEQ |
|---|---|---|
| primer | | ID |
| ID | primer sequence | NO: |
| A3B01F | 5′-TCGTATCGTGAGCGTCAACCGACTGAGCATAA- | 42 |
| | AAAACGAGGGTTGTCAAAGACCCCA-3 ′ | |
| A3B02F | 5′-TTAGTCACTGTACAGCGTGAGGCGTAGTTCAC- | 43 |
| | AAAACGAGGGTTGTCAAAGACCCCA-3 ′ | |
| A3B03F | 5′-ATCAGTACGTTGCTAGCTTGAGCGATAGCCAG- | 44 |
| | AAAACGAGGGTTGTCAAAGACCCCA-3 ′ | |
| A3B04F | 5′-AGTATGCACGACCGGATCTGTCAACGATACGT- | 45 |
| | AAAACGAGGGTTGTCAAAGACCCCA-3 ′ | |
| A3B05F | 5′-GAAGTCTAGATCAATCGTTAGCATCTGCTCGC- | 46 |
| | AAAACGAGGGTTGTCAAAGACCCCA-3 ′ | |
| A3B06F | 5′-AGACGTCTGACGATGCTCATAACCTGGACATC- | 47 |
| | AAAACGAGGGTTGTCAAAGACCCCA-3 ′ | |
| A3B07F | 5′-CTCATTCGATGTATGTCGCAGGTAGCAAGCAT- | 48 |
| | AAAACGAGGGTTGTCAAAGACCCCA-3 ′ | |
| A3B08F | 5′-TACTGTCGATTCGACCACGACTAGGCTATGCT- | 49 |
| | AAAACGAGGGTTGTCAAAGACCCCA-3 ′ | |
| A3B09F | 5′-CTGTACTCCGATGAACGGCGATCTAGTCTACG- | 50 |
| | AAAACGAGGGTTGTCAAAGACCCCA-3 ′ | |
| A3B10F | 5′-CGATGGTACTCAGATCGGCGACATCAGTTGAT- | 51 |
| | AAAACGAGGGTTGTCAAAGACCCCA-3 ′ | |
| A3B11F | 5′-AGTGCTAGTCGATGCCGCTGCATACCTATGAT- | 52 |
| | AAAACGAGGGTTGTCAAAGACCCCA-3 ′ | |
| A3B12F | 5′-ACATCTTACGGCTCGACTGGCACGATGTCTGA- | 53 |
| | AAAACGAGGGTTGTCAAAGACCCCA-3 ′ | |
| A3B13F | 5′-ACTTAAGTCGAGTCGCATGCCTGTCGCTAGAT- | 54 |
| | AAAACGAGGGTTGTCAAAGACCCCA-3 ′ | |
| A3B14F | 5′-ATCAAGATGTACCACGTCAGGCTAGTACTGCT- | 55 |
| | AAAACGAGGGTTGTCAAAGACCCCA-3 ′ | |
| A3B15F | 5′-TCGAGCTTCGAGTGATAACGTAACGCTGCGTA- | 56 |
| | AAAACGAGGGTTGTCAAAGACCCCA-3 ′ | |
| A3B16F | 5′-GCAGATGACCACTACGTCGAACTGACTTGACT- | 57 |
| | AAAACGAGGGTTGTCAAAGACCCCA-3 ′ | |
| A3B17F | 5′-TCAGCATAGCGTCGATCACCAATGCATGCTAG- | 58 |
| | AAAACGAGGGTTGTCAAAGACCCCA-3 ′ | |
| A3B18F | 5′-CTCGATGACAGATGCGATACTGGCGTTCAATG- | 59 |
| | AAAACGAGGGTTGTCAAAGACCCCA-3 ′ | |
| A3B19F | 5′-GCGTCAGCTACGATTGTATCCAAGTGCTCGAT- | 60 |
| | AAAACGAGGGTTGTCAAAGACCCCA-3 ′ | |
| A3B20F | 5′-GACATTGACTGCTATGACGCCTTGAGTAGCAG- | 61 |
| | AAAACGAGGGTTGTCAAAGACCCCA-3 ′ | |
| A3B21F | 5′-AGCAACGCTAGTGGCCGCTATGTACTAGCTCG- | 62 |
| | AAAACGAGGGTTGTCAAAGACCCCA-3 ′ | |
| A3B22F | 5′-TACATCTGGCGAGTATGATCCTACGGTGAGTC- | 63 |
| | AAAACGAGGGTTGTCAAAGACCCCA-3 ′ | |
| A3B23F | 5′-ATCAGCTGTTACGATAGGCGACTCGCCATCGA- | 64 |
| | AAAACGAGGGTTGTCAAAGACCCCA-3 ′ | |
| A3B24F | 5′-CGCACTAGTATCAGCCTAAGCACTCGTGATGG- | 65 |
| | AAAACGAGGGTTGTCAAAGACCCCA-3 ′ | |
| A3B01R | 5′-TCGTATCGTGAGCGTCAACCGACTGAGCATAA- | 66 |
| | ACAAACCTGATAACGCAAGCTACTGC-3′ | |
| A3B02R | 5′-TTAGTCACTGTACAGCGTGAGGCGTAGTTCAC- | 67 |
| | ACAAACCTGATAACGCAAGCTACTGC-3′ | |
| A3B03R | 5′-ATCAGTACGTTGCTAGCTTGAGCGATAGCCAG- | 68 |
| | ACAAACCTGATAACGCAAGCTACTGC-3′ | |
| A3B04R | 5′-AGTATGCACGACCGGATCTGTCAACGATACGT- | 69 |
| | ACAAACCTGATAACGCAAGCTACTGC-3′ | |
| A3B05R | 5′-GAAGTCTAGATCAATCGTTAGCATCTGCTCGC- | 70 |
| | ACAAACCTGATAACGCAAGCTACTGC-3′ | |
| A3B06R | 5′-AGACGTCTGACGATGCTCATAACCTGGACATC- | 71 |
| | ACAAACCTGATAACGCAAGCTACTGC-3′ | |
| A3B07R | 5′-CTCATTCGATGTATGTCGCAGGTAGCAAGCAT- | 72 |
| | ACAAACCTGATAACGCAAGCTACTGC-3′ | |
| A3B08R | 5′-TACTGTCGATTCGACCACGACTAGGCTATGCT- | 73 |
| | ACAAACCTGATAACGCAAGCTACTGC-3′ | |
| A3B09R | 5′-CTGTACTCCGATGAACGGCGATCTAGTCTACG- | 74 |
| | ACAAACCTGATAACGCAAGCTACTGC-3′ | |
| A3B10R | 5′-CGATGGTACTCAGATCGGCGACATCAGTTGAT- | 75 |
| | ACAAACCTGATAACGCAAGCTACTGC-3′ | |
| A3B11R | 5′-AGTGCTAGTCGATGCCGCTGCATACCTATGAT- | 76 |
| | ACAAACCTGATAACGCAAGCTACTGC-3′ | |
| A3B12R | 5′-ACATCTTACGGCTCGACTGGCACGATGTCTGA- | 77 |
| | ACAAACCTGATAACGCAAGCTACTGC-3′ | |
| A3B13R | 5′-ACTTAAGTCGAGTCGCATGCCTGTCGCTAGAT- | 78 |
| | ACAAACCTGATAACGCAAGCTACTGC-3′ | |
| A3B14R | 5′-ATCAAGATGTACCACGTCAGGCTAGTACTGCT- | 79 |
| | ACAAACCTGATAACGCAAGCTACTGC-3′ | |
| A3B15R | 5′-TCGAGCTTCGAGTGATAACGTAACGCTGCGTA- | 80 |
| | ACAAACCTGATAACGCAAGCTACTGC-3′ | |
| A3B16R | 5′-GCAGATGACCACTACGTCGAACTGACTTGACT- | 81 |
| | ACAAACCTGATAACGCAAGCTACTGC-3′ | |
| A3B17R | 5′-TCAGCATAGCGTCGATCACCAATGCATGCTAG- | 82 |
| | ACAAACCTGATAACGCAAGCTACTGC-3′ | |
| A3B18R | 5′-CTCGATGACAGATGCGATACTGGCGTTCAATG- | 83 |
| | ACAAACCTGATAACGCAAGCTACTGC-3′ | |
| A3B19R | 5′-GCGTCAGCTACGATTGTATCCAAGTGCTCGAT- | 84 |
| | ACAAACCTGATAACGCAAGCTACTGC-3′ | |
| A3B20R | 5′-GACATTGACTGCTATGACGCCTTGAGTAGCAG- | 85 |
| | ACAAACCTGATAACGCAAGCTACTGC-3′ | |
| A3B21R | 5′-AGCAACGCTAGTGGCCGCTATGTACTAGCTCG- | 86 |
| | ACAAACCTGATAACGCAAGCTACTGC-3′ | |
| A3B22R | 5′-TACATCTGGCGAGTATGATCCTACGGTGAGTC- | 87 |
| | ACAAACCTGATAACGCAAGCTACTGC-3′ | |
| A3B23R | 5′-ATCAGCTGTTACGATAGGCGACTCGCCATCGA- | 88 |
| | ACAAACCTGATAACGCAAGCTACTGC-3′ | |
| A3B24R | 5′-CGCACTAGTATCAGCCTAAGCACTCGTGATGG- | 89 |
| | ACAAACCTGATAACGCAAGCTACTGC-3′ | |

Barcoded primers for amplicon-3. The left part of the
sequence is the barcode. F: forward; R: reverse.

### Barcoded Long-Range PCR to Amplify the Region Flanking Exon-1 of the HTT Gene

For the French HD cohort, the target region was covered by two overlapping amplicons (amplicon-1 and amplicon-2, FIG. 1F). Amplicon-1 mainly covers the upstream region of the CAG repeat region (GRCh38, chr4: 3069608-3075517). The amplicon length (without barcode) is 5910 bp. Amplicon-2 mainly covers the downstream region of the CAG repeat region (GRCh38, chr4: 3071119-3079972). The amplicon length (without barcode) is 8854 bp. For each amplicon, ten different barcodes were added to the 5′-side of both forward and backward primers. The complete primer sequences are listed in Table 2 and Table 3. The combination of both forward and backward primers can be used to multiplex 100 samples. The French HD cohort contains 396 HD genomic DNA samples which were stored in five 96-well plates where plates 1~4 each contains 95 samples and plate 5 contains 16 samples. For each plate of plates 1-4, barcoded PCR was performed with 95 barcode combinations, and the pooled samples were then sequenced in one Nanopore flow cell. Samples in plate 5 were sequenced together with the QC-failed samples of plates 1-4. The long-range PCR was performed using the PrimeSTAR® GXL DNA Polymerase (Takara Bio USA, San Jose, CA). The PCR condition was 1) 95° C. for 3 minutes; 2) 98° C. for 10 seconds; 3) 68° C. for 10 minutes. Steps 2 to 3 were repeated for 30 cycles.

For the CHDI HD cohort, the target region was covered by one single amplicon (amplicon-3, FIG. 1F). The forward primer is the forward primer of amplicon-1 and the backward primer is the backward primer of amplicon-2. The amplicon length (without barcode) is 10365 bp. 24 different barcodes were added to the 5′-side of both forward and backward primers. The combination of both forward and backward primers can be used to multiplex 576 samples. The CHDI HD cohort contains 960 samples, which were stored in ten 96-well plates. 480 samples (five plates) were multiplexed at a time. The PCR condition and sequencing process are the same as the French HD cohort.

### Long-Read Sequencing of the Barcoded PCR Products

The barcoded PCR products were pooled and purified with Agencourt® AMPure® XP beads (Beckman Coulter, A63881). The sequencing library was prepared using the ligation sequencing kit (ONT, SQK-LSK109), according to manufacturer's instructions. 1) DNA repair, end repair, and dA-tailing. 1 μg of the pooled and purified PCR products were used as input DNA, and the volume was adjusted to 47 μl with nuclease-free water. 1 μl DNA CS (ONT, SQK-LSK109), 47 μl input DNA, 3.5 μl NEBNext® FFPE DNA Repair Buffer (NEB, M6630), 2 μl NEBNext® FFPE DNA Repair Mix (NEB, M6630), 3.5 μl Ultra™ II End-prep reaction buffer (NEB, E7546), 3 μl Ultra™ II End-prep enzyme mix (NEB, E7546) were mixed in a PCR tube. The mixture was incubated at 20° C. for 5 minutes and 65° C. for 5 minutes. A 1× volume (60 μl) AMPure® XP clean-up was performed, and the DNA was eluted in 61 μl nuclease-free water. A 1-μl aliquot was quantified by fluorometry (Qubit) to ensure ≥700 ng DNA was retained. 2) Adapter ligation. 60 μl DNA sample from the previous step, 25 μl Ligation Buffer (ONT, SQK-LSK109), 10 μl NEBNext® Quick T4 DNA Ligase (NEB, E6056), and 5 μl Adapter Mix (ONT, SQK-LSK109) were mixed in order. The mixture was incubated for 10 minutes at room temperature. The adaptor-ligated DNA was cleaned up by adding a 0.4× volume (40 μl) of AMPure® XP beads, incubating for 5 minutes at room temperature, and resuspending the pellet twice in 250 μl Long Fragment Buffer (ONT, SQK-LSK109). The purified-ligated DNA was resuspended in 15 μl elution buffer (ONT, SQK-LSK109). A 1-μl aliquot was quantified by fluorometry (Qubit) to ensure ≥300 ng DNA was retained. 50 fmol (278 ng) of this final prepared library was loaded onto the GridION™ sequencer with an FLO-MIN106D (R9.4.1) flow cell. The sequencing was run for 48 hours. Live basecalling was performed using the MinKNOW™ software.

Demultiplexing of the Sequencing Data with NanoBinner

NanoBinner, a tool for de-multiplexing of barcoded amplicons from long-read sequencing data, was developed (github.com/WGLab/NanoBinner). Given the moderate error rate in long reads, there might be random matches of barcodes inside the amplicon sequence. To avoid this potential issue, NanoBinner aligns the barcode sequence as well as 256 bp amplicon sequence next to it. The 256 bp amplicon sequence acts as an anchor so that the matching of barcodes is at the correct position. The alignment is performed using minimap2 (Li, H. (2018) Bioinformatics 34:3094-3100) with the parameter for nanopore reads (−x map-ont). This parameter can be changed if the input is PacBio reads. Minimap2 calculates a mapping quality score for each barcode, which is the Phred scale of the probability that a read is misplaced. NanoBinner assigns a read to a barcode if the Phred scale mapping quality score was ≥30. Here, the combination of the two barcodes on both sides determines the sample. A read was assigned to a specific sample if the barcodes on both sides were confidently determined. One FASTQ file was generated for each sample.

Repeat Detection and Read Phasing with NanoRepeat

NanoRepeat (github.com/WGLab/NanoRepeat) can quantify a single tandem repeat or jointly quantify two adjacent repeats. In this study, the CAG and CCG repeats were jointly quantified in the HTT gene. The joint quantification process has two steps: fast estimation and refining. In the fast estimation step, NanoRepeat performs a quick analysis of each repeat and estimates the lower and upper bound of the repeat size independently. This analysis is done by aligning the reads to a decoy reference sequence with 1000 repeat units (CAG or CCG) using minimap2 (Li, H. (2018) Bioinformatics 34:3094-3100). Given the sequencing error rate, the alignment has some tolerance of mismatches and thus, the nearby non-repeat region in the reads might be forced to be aligned with the decoy reference sequence. Therefore, the upper bound of the repeat size is the number of aligned repeat units in the decoy reference sequence. The lower bound of the repeat size was assigned as the number of repeat units in the read that exactly matched the reference sequence with no error. Let L₁, L₂ denote the lower bounds of the CAG and CCG repeats, and U₁, U₂ denote the upper bounds of the two repeats, respectively. In the refining step, NanoRepeat generates a batch of amplicon sequences with m(L₁≤m≤U₁) CAG repeat units and n(L₂≤n≤U₂) CCG repeat units (FIG. 1C). Each read is aligned to this batch of amplicon sequences with minimap2 (Li, H. (2018) Bioinformatics 34:3094-3100). The m and n that maximize the alignment score were the estimated CAG and CCG repeat sizes of the read.

After the repeat number of each read was determined, NanoRepeat classifies the reads to alleles. First, outlier reads with repeat sizes that are outside three standard deviations from the mean were removed. Next, it was assumed that the CAG and CCG repeat sizes (m, n) are distributed according to a mixture of N Gaussian models, where Nis 1 or 2 as human is a diploid genome. Akaike information criterion (AIC) or Bayesian information criterion (BIC) are commonly used criteria to select the best value of N and prevent overfitting. As Gaussian distribution is a probability distribution of real-valued random variables, it was found that these two methods are not able to prevent overfitting of the GMM if the input random variables are rounded to integers. Therefore, a uniform distributed random noise (between-0.5 and 0.5) was added to each m and n before model selection using BIC. After the best Nis selected, the original value of m and n (without the random noise) was used to train the GMM. The label of each read was predicted using the trained model. To make sure the subsequent SNP calling is accurate, a read is discarded if it is not within 95% equi-probability surface of a Gaussian model. A sample failed QC if only one allele was detected (N=1) or one of the alleles had less than 50 reads.

### SNP/Indel Detection

To reduce the computational time, the reads of each allele were down-sampled to 200× coverage and then error-corrected by Canu (version: 2.0) (Koren, et al. (2017) Genome Res., 27:722-736). The error-corrected reads were aligned to human reference genome GRCh38 using minimap2 (Li, H. (2018) Bioinformatics 34:3094-3100) with the parameter for nanopore reads (−x map-ont). The SAM file was converted to BAM file and sorted by SAMtools (Li, et al. (2009) Bioinformatics 25:2078-2079). SNP/Indel calling was performed using longshot (Edge, et al. (2019) Nat. Commun., 10:4660) with default parameters. Homozygous calls of each allele were considered accurate and were used in downstream analysis.

### Generation of Consensus Sequence and SV Detection

The error-corrected reads generated during SNP/Indel detection were assembled by Canu (version: 2.0) (Koren, et al. (2017) Genome Res., 27:722-736), and a consensus sequence was generated for each chromosome. The consensus sequence was aligned to the reference genome GRCh38 using minimap2 (Li, H. (2018) Bioinformatics 34:3094-3100) with the parameter for assembly contigs (−x asm20). SVs were called directly from the alignment using a custom pipeline.

### Analysis of Gain and Loss of PAMs Mediated by SNPs

An SNP is considered to mediate the gain of a PAM if the alternative allele contains a high-efficiency PAM and the reference allele does not contain any high-efficiency PAM or low-efficiency PAM of the same CRISPR enzyme. Conversely, an SNP is considered to mediate the loss of a PAM if the reference allele contains a high-efficiency PAM and the alternative allele does not contain any high-efficiency PAM or low-efficiency PAM. TTTN is a high-efficiency PAM of AsCpf1. Both NGG and NAG are considered high-efficiency PAMs of the wild-type SpCas9, although the recognition for NAG is less efficient than NGG. For other Cas9 enzymes of which the post-selection PAM depletion values (PPVDs) are measured (Kleinstiver, et al. (2015) Nature 523:481-485), a PAM is considered as a high-efficiency PAM if the PPVD is less than 0.2 (fivefold depletion). The list of high-efficiency and low-efficiency PAMs analyzed in this study is shown in Table 5.

TABLE 4

| | High efficiency PAMs | | |
|---|---|---|---|
| Enzyme | (included) | Low efficiency PAMs (excluded) | Reference |
| SpCas9 | NGG, NAG | — | — |
| SpCas9_VQR | NGAG, NGAT, NGAC, | NGGA, NGGT, NGGC | (1) |
| | NGAA, NGCG, NGTG, | | |
| | NGGG, NAAG | | |
| SpCas9_EQR | NGAG, NGCG, NGAT, | NGGG, NGTG, NGAC | (1) |
| | NGAA | | |
| SpCas9_VRER | NGCG | — | (1) |
| SaCas9 | NGGGT, NGAAT, NGAGT, | NGGAA, NGGAC, NGACT, NGGCA, | (1) |
| | NGGAT | NGGGA, NGATC, NGGAG, NGATT, | |
| | | NGGTA, NGGTT, NGACA, NGATA, | |
| | | NGGGC, NGGGG, NGAGC, NGACC, | |
| | | NGAAG, NGGCT, NGCAT, NGACG, | |
| | | NGCGT, NGCAC, NGATG, NAAGT, | |
| | | NAGGT, NGGCG, NGTTT, NGGCC, | |
| | | NGCTT, NCAGT, NGCGA, NGGTG, | |
| | | NGGTC, NCGAT, NGCAA, NAGAT, | |
| | | NGTGT | |
| AsCpf1 | TTTN | — | (2) |

CRISPR enzymes and PAMs analyzed in this study. (1) Kleinstiver et al.
(2015) Nature 523(7561): 481-5. (2) Zetsche, et al. (2015) Cell 163(3): 759-71.

### Sanger Sequencing to Validate SNP16 (Rs3856973)

A 526 bp region flanking SNP16 (rs3856973, chr4: 3078446G>A on GRCh38) was amplified by a nested PCR, as a specific primer that directly amplifies this region was not designed. In the first round of the nested PCR, the primers of amplicon-2 (Table 3) were used, which amplified an 8.8 kb region. The PCR condition was described in the above section. 1 μl of the PCR product was used as the input of the second round of PCR. In the second round, the primers were 5′-TTGGGAGGGTCCTCACAGTA-3′ (forward; SEQ ID NO: 90) and 5′-GAGGTTGCAGTGAGCCAAGA-3′ (backward; SEQ ID NO: 91). The PCR condition was 1) 95° C. for 3 minutes; 2) 98° C. for 10 seconds; 3) 68° C. for 45 seconds (30 cycles). Sanger sequencing was performed using the forward primer of the second-round PCR.

### TaqMan® SNP Genotyping Assay to Validate SNP16

The TaqMan® Genotyping Master Mix (cat #: 4371353) and the probes for SNP16 (Assay ID: C_27529960_10) were ordered from Thermo Fisher Scientific. A 5 μl PCR reaction system was used. Real-time PCR and data analysis were performed following the manufacturer's instructions.

### Cell Culture and Transfection

Human embryonic kidney (HEK 293) cells (obtained from CHOP Research Vector Core stock) were maintained in DMEM media containing 10% Fetal Bovine Serum (FBS), 1% L-Glutamine and 1% penicillin/streptomycin at 37° C. with 5% CO₂. Cells were cultured in 24 well plates and transfected at 80-90% confluence using Lipofectamine™ 2000 transfection reagent, according to the manufacturer's protocol. After DNA transfection, cells expressing SaCas9 and sgRNA sequences were enriched by puromycin selection (3 μM) for 24 hours, and subsequently expanded for genomic DNA and RNA extraction. Human HD fibroblasts (obtained from Coriell Institute for Medical Research cell repository) were maintained on DMEM media supplemented with 10% Fetal Bovine Serum (FBS), 1% MEM non-essential amino acids, 1% penicillin/streptomycin and 1% L-Glutamine at 37° C. with 5% CO₂. DNA transfection was done by electroporation using Invitrogen Neon transfection reagent using the electroporation conditions (ND33392: 1450V, 20 ms, 2 pulses), following the guidelines provided by the manufacturer. Fibroblasts were selected with puromycin (2 μM) for 24 hours and subsequently expanded for genomic DNA extraction. Cells were not authenticated or tested for Mycoplasma by the investigators since they previously passed the quality controls of CHOP Research Vector Core and the Coriell Institute for Medical Research cell repository. None of the cells used in the study were listed in ICLAC database of commonly misidentified cell lines.

sgRNA and Cas9 Plasmid Construction

The plasmid pX330 containing the SpCas9 and sgRNA expression cassettes (Monteys, et al. (2017) Mol. Ther., 25:12-23) was used as a template to clone the SaCas9 cDNA and sgRNA sequences. To determine transfection efficacy and for selecting positively transfected cells, a CMV reporter cassette expressing eGFP/P2A/Puromicin fusion protein was cloned downstream of the SaCas9 expression cassette. For all sgRNAs, the guide complementary sequences were cloned using a single cloning step with a pair of partially complementary oligonucleotides. The oligo pairs encoding the genomic complementary guide sequences were annealed and ligated into the BbsI cloning site upstream and in frame with the invariant scaffold of the sgRNA sequence. The gRNA sequences for targeting SNP1, SNP16 and HDi3 are: GCCCCGCTCCAGGCGTCGGCG (SNP1; SEQ ID NO: 95), GATAGGGAAATGTCAGGGTTAA (SNP16; SEQ ID NO: 134) and GTGCTTTTAGGACGCCTCGGC (Hdi3; SEQ ID NO: 128).

RNA Extraction and qRT-PCR

Total RNA was extracted using Trizol™ (Life Technologies) according to the manufacturer's protocol, with the exception of 1 μL of Glycoblue™ (Life Technologies) in addition to the aqueous phase on the isopropanol precipitation step and a single wash with cold 70% ethanol. RNA samples were quantified by spectrophotometry and subsequently cDNAs generated from 1 μg of total RNA with random hexamers (TaqMan® RT reagents, Applied Biosystems). To determine human HTT expression levels in HEK293 cells, TaqMan® probes was used for human HTT and glyceraldehyde 3-phosphate dehydrogenase (GAPDH) mRNAs obtained from Applied Biosystems. Relative HTT gene expression was determined using the ddCt method.

### Semiquantitative PCR for Assessment of Allele-Specific Editing

Allele-specific editing was assessed by semiquantitative PCR amplification of the CAG repeat within HTT exon-1. Genomic DNA (gDNA) was extracted from cultured HD fibroblasts using a QiaAMP DNA mini kit (QIAGEN) according to manufacturer's instructions. The gDNA was quantified by fluorometry (Qubit) and then diluted to the same concentration (5 ng/μl). BIOLASE™ DNA Polymerase (Bioline) was used to amplify the input DNA templates. As the CAG repeat and its flanking region has a high GC content (72.4%), Betaine was added to the PCR reaction system to enhance amplification. The 50 μl PCR reaction system contains 8 μl gDNA (5 ng/μl), 5 μl 10× NH₄ buffer, 2 μl dNTP mixture (2.5 mM each), 1.5 μl MgCl₂ (50 mM), 1 μl BIOLASE DNA Polymerase, 12 μl Betaine (5M), 1 μl primer mixture (10 μM each), and 19.5 μl ddH₂O. The PCR thermal cycling program was 95° C. for 2 minutes, followed by 25 cycles of 95° C. for 15 seconds, 57° C. for 15 seconds, 72° C. for 45 seconds, and a final extension at 72° C. for 5 minutes. The PCR products were separated by electrophoresis through a 1.5% agarose gel stained with ethidium bromide. The gel bands were quantified using the Image Lab software (Bio-Rad).

### Statistical Analysis

Statistical analyses were performed using GraphPad Prism v7 software. Outlier samples were detected using the Grubb's test (a=0.05). Normal distribution of the samples was determined by using the D'Agostino and Pearson normality test. Data was analyzed using one-way ANOVA followed by a Bonferroni's post hoc. Statistical significance was considered with a p<0.05. All results are shown as the mean±SEM.

### Results

### Sequencing and Data Analysis Workflow Overview

The workflow of this study is shown in FIG. 1. Barcoded long-range PCR was used to amplify the target regions. The barcoded sequences were custom-designed such that: 1) there were neither tandem repeat sequences nor sequences similar to the human genome; 2) the pairwise sequence similarity between each two barcodes was minimized; 3) the GC content is between 40-60% (FIG. 1G). For both HD cohorts, the same target region (GRCh38, chr4: 3069608-3079972), which begins 5268 bp upstream of the CAG repeat and ends 5096 bp downstream, was amplified (FIG. 1F). A combinatorial barcoding strategy, which adds different barcodes 5′- of both forward and backward primers, was used (Tables 2-4). The barcoded amplicons were pooled, size selected and then sequenced in a MinION™ flow cell using the Oxford Nanopore GridION™ sequencer.

The sequencing data was de-multiplexed with NanoBinner, which was originally developed for this work. Given the moderately high error rate and the flexibility of alignment for long reads, there may be random barcode matches inside the amplicon sequence. To avoid potential random matching, NanoBinner compares the barcode sequence as well as the 256 bp amplicon sequence immediately next to the barcode. The 256 bp amplicon sequence acts as an anchor such that the matching of barcodes is at the correct position. NanoBinner also uses a new algorithm to assign barcodes.

After de-multiplexing, CAG repeat sizes are detected by NanoRepeat, a novel repeat detection tool originally developed for this work. Through use of a novel algorithm, it jointly quantifies two adjacent tandem repeats, phases the reads using repeat sizes from both repeats and reports haplotypes. In exon-1 of HTT, there is a CCG repeat immediately 3′ of the CAG repeat, with (CCG) 7 (SEQ ID NO: 92) and (CCG) 10 (SEQ ID NO: 93) predominant although there are other variants (Andrew, et al. (1994) Hum. Mol. Genet., 3:65-67; Pramanik, et al. (2000) Eur. J. Hum. Genet., 8:678-682; Agostinho Lde, et al. (2012) J. Hum. Genet., 57:796-803). The joint quantification of the CAG and CCG repeat has two steps. First, NanoRepeat performs a quick analysis of each repeat and independently determines the lower bound and upper bound of the repeat size. Next, NanoRepeat generates a batch of amplicon sequences with m(L₁≤Sm≤U₁) CAG repeat units and n(L₂≤n≤U₂) CCG repeat units, where L₁, L₂ are the lower bounds of the two repeats and U₁, U₂ are the upper bounds of the two repeats, respectively (FIG. 1C). Each read is aligned to this batch of amplicon sequences using minimap2 (Li, H. (2018) Bioinformatics 34:3094-3100). The m and n that maximize the alignment score are the estimated CAG and CCG repeat sizes of the read. After the joint quantification, NanoRepeat uses the Gaussian mixture model (GMM) to group the reads to alleles. Outlier reads are removed so that the subsequent SNP calling is accurate. At least 50 reads per allele are required for SNP calling. The reads assigned to each allele were error-corrected by Canu (Koren, et al M. (2017) Genome Res., 27:722-736), with SNPs/indels called by longshot (Edge, et al. (2019) Nat. Commun., 10:4660).

### Assessment of Computational Methods

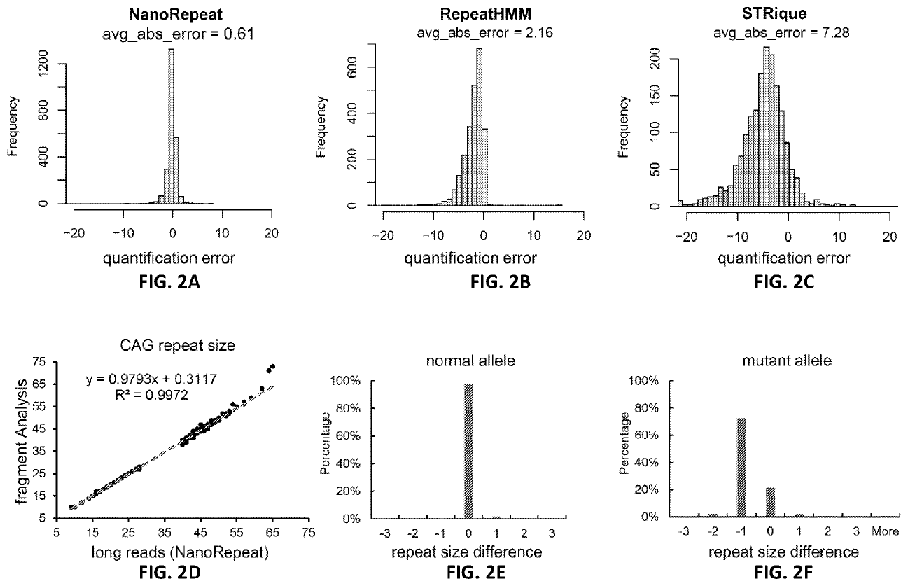

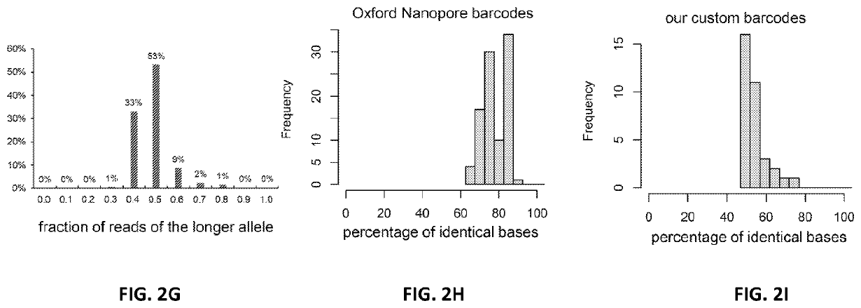

The accuracy of repeat quantification in whole-genome sequencing data was tested. The Oxford Nanopore dataset of the CHM13 genome was used (Nurk, et al. (2022) Science 376:44-53). The CHM13 cell line has near-complete homozygosity, with only a few exceptions. The telomere-to-telomere consortium has finished the de novo assembly of CHM13 primarily based on PacBio HiFi reads, but supplemented with data from other sequencing platforms. As the CHM13 v1.1 assembly sequence is highly reliable, repeat counts from the assembly sequence can be considered as a truth set for method assessment. NanoRepeat was benchmarked along with two other widely-used repeat detection tools, namely RepeatHMM (Liu, et al. (2017) Genome Med., 9:65) and STRique (Giesselmann, et al. (2019) Nat. Biotechnol., 37:1478-1481). Repeat detection was performed on 2,370 short tandem repeat (STR) regions in the CHM13 genome. These are all STR regions that are >100 bp and not within a 500 bp flanking region of another STR. Adjacent STRs were removed because many of the adjacent STRs have similar sequences and it is hard to tell if they need to be merged or not without manual examination. The length of the 2,370 STR regions range from 100 bp to 2374 bp. The evaluation results are shown in FIGS. 2A-C. NanoRepeat has much smaller quantification error compared with RepeatHMM and STRique. In addition, RepeatHMM and STRique have a systematic quantification bias which leads to underestimation of the repeat size.

Next, repeat detection was assessed in the HD samples. The CHDI Foundation provided the repeat sizes quantified earlier by PCR-based Fragment Analysis. The repeat size quantified by NanoRepeat and the data provided by the CHDI Foundation were compared. As shown in FIG. 2D, NanoRepeat's quantifications based on the long reads are highly consistent with the results of Fragment Analysis provided earlier (R²=0.9972). For normal alleles, the two methods were identical for approximately 98% of samples (FIG. 2E). For expanded alleles, repeat size differences between the two methods were within one repeat unit for more than 95% samples (FIG. 2F). There is very little bias between the normal and the expanded alleles (FIG. 2G).

The custom-designed barcodes were compared with the 96 barcodes provided by Oxford Nanopore Technology (ONT). 35 of the 96 barcodes in ONT's PCR Barcoding Expansion Pack can be aligned to the human reference genome GRCh38 with more than 80% identical bases (FIG. 2H). For example, BC71 is 24 bp and can be aligned to chr18: 46568247-46568269 (GRCh38) with 22 bp matched. To avoid potential non-specific binding in the PCR reaction, custom barcodes were designed. As shown in FIG. 2I, none of the custom barcodes has more than 80% identical bases in the human reference genome GRCh38.

### QC Summary of the Amplicon Sequencing Experiments

In total, 318 samples from the French HD consortium and 664 samples from the CHDI Foundation passed QC and had sufficient sequencing data to make reliable variant calls. To ensure accurate SNP detection, a stringent QC criterion was required: at least 50× coverage for each allele. The QC summary of the amplicon sequencing experiments is shown in Table 1. The French cohort was amplified twice with different primers. Amplicon-1 (5.9 kb) mainly covers the upstream region of exon-1 while amplicon-2 (8.8 kb) mainly covers the downstream region. The two regions have some overlap and both covers the CAG repeat region. Therefore, a full-length haplotype can be assembled from phased SNPs of the two amplicons. For amplicon-1, 93% (370/396) of the samples passed QC while 90% (355/396) of the samples passed QC for amplicon-2. 88% (348/396) samples passed QC for both amplicons and were used to assemble the full-length haplotype.

TABLE 1
Quality control summary of the amplicon sequencing experiments.

| Cohort | French | French | CHDI |
|---|---|---|---|
| total # of samples | 396 | 396 | 960 |
| amplicon | Amplicon-1 | Amplicon-2 | Amplicon-3 |
| amplicon length (bp) | 5910 | 8854 | 10365 |
| # of pooled samples | 100 | 100 | 480 |
| per flow cell | | | |
| QC-passed samples | 370 | 355 | 708 |
| pass rate (%) | 93% | 90% | 74% |

The CHDI cohort was amplified using the forward primer of amplicon-1 and backward primer of amplicon-2. This amplicon is referred to as amplicon-3 (10.3 kb, FIG. 1F). 74% (708/960) samples passed QC. This rate is lower than that of the French cohort, probably due to 1) the length of amplicon-3 is longer than amplicon-1 and amplicon-2, thus amplicon-3 is more difficult to amplify and requires higher DNA integrity; 2) some samples are of low concentration. Therefore, the French cohort was used as the main cohort for analysis and validate the conclusions on the CHDI cohort.

CAG and CCG Repeat Sizes Detected from HD Samples

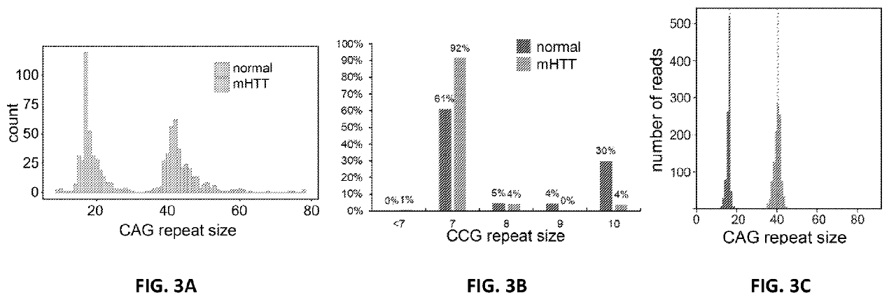

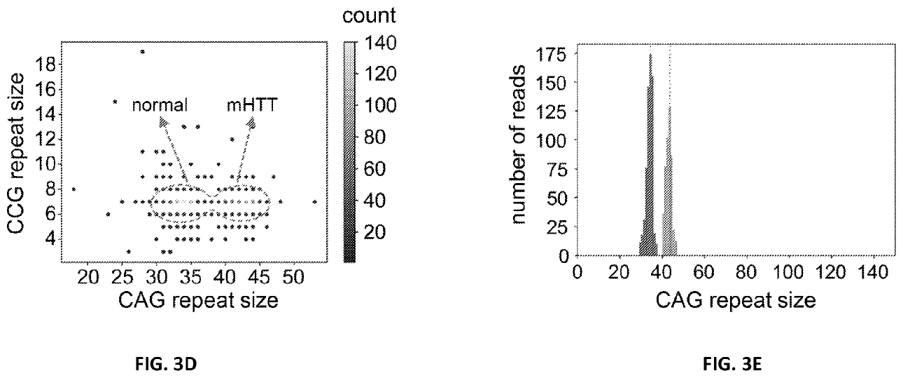

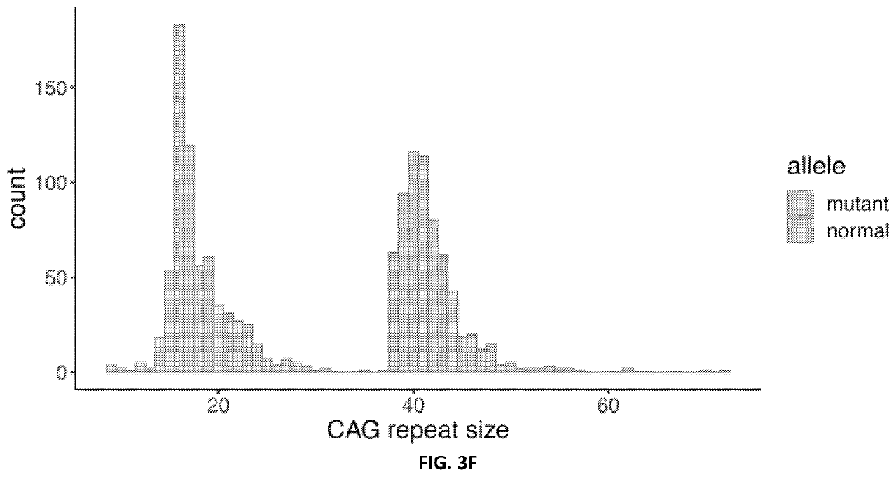

The distribution of CAG repeat size is shown in FIG. 3A (French cohort) and FIG. 3F (CHDI cohort). Alleles with ≤35 CAG repeat units are denoted as normal alleles, and those with ≥36 CAG repeat units denoted as expanded alleles. Most normal alleles are less than 26 repeats; repeat sizes between 27 and 35 were rare, consistent with other studies (Warby, et al. (2009) Am. J. Hum. Genet., 84:351-366). CCG repeat size distribution were different among normal and mHTT chromosomes (FIG. 3B). For normal, there were two predominant alleles: (CCG)₇ (SEQ ID NO: 92) and (CCG)₁₀ (SEQ ID NO: 93). In mHTT chromosomes, (CCG) 7 predominates (~92%).

Since there was often a large difference (>20) in CAG repeat size between the normal and mHTT chromosomes, it was easy to separate the reads derived from normal and mHTT alleles for most samples (FIG. 3C). One sample had CAG repeat sizes of 34 (intermediate) and 43 (expanded), which is the smallest CAG repeat size difference in the French HD cohort; both alleles have the same CCG repeat size. With the Gaussian mixture models, it was possible to compute the probabilities and remove the reads that were not confidently classified so that the two alleles were well separated (FIGS. 3D-3E).

SNP with High Allele Frequency in HD Samples

After the reads were phased according to the CAG and CCG repeats, SNP detection was performed for each allele, generating a list of haplotyped SNPs of 10.3 kb. The complete list of the SNPs is shown in Table 6. A total of 110 SNPs were identified from the two HD cohorts; 56 SNPs are in the upstream HTT gene region; one SNPs is in 5′-UTR; 7 SNPs are in the exon-1 coding region and 46 SNPs are in intron-1. 8 SNPs are novel and have not been found in dbSNP, the Genome Aggregation Database (gnomAD) or the 1000 Genomes Project (Karczewski, et al. (2020) Nature 581:434-443). The 8 novel SNPs were validated by examining the alignments using the Integrative Genomics Viewer (IGV; Robinson, et al. (2011) Nat. Biotechnol., 29:24-26), such novel SNPs could be still due to errors in long range PCR and subsequent sequencing.

The SNPs identified from the French cohort were analyzed. Among those identified, 19 are common with allele frequencies (AFs) of ≥5% in the normal or mHTT chromosomes, or the gnomAD database (non-Finnish European population). The positions of the 19 SNPs are shown in FIG. 4A. Notably, their allele frequencies (AFs) in normal and mHTT chromosomes are dramatically different (FIGS. 4B, 4E). To verify SNP detection, the AFs of all identified SNPs were compared with their AFs in the gnomAD database. The AFs of SNPs in the normal chromosomes are highly correlated (R²=0.9725) with their AFs in non-Finnish European (NFE) population in the gnomAD database. However, the correlation between the AFs of SNPs in the mHTT chromosomes and those in the gnomAD database are low (R²=0.3936) (FIGS. 4C-4D). This indicates unique haplotypes for mHTT chromosomes, which is consistent with findings that CAG expansion events are associated with specific haplogroups (Warby, et al. (2009) Am. J. Hum. Genet., 84:351-366). Examination of the AFs of SNPs identified from the CHID cohort validated the findings.

### Identification of SNPs for Allele-Specific Genome Editing

The gain and loss of PAMs mediated by the 19 common SNPs was analyzed as they comprise the majority of diversity in HD individuals and the general population, yielding putative targets for allele-specific genome editing. In addition to the canonical PAMs (NGG and NAG) recognized by SpCas9, PAMs for 5 other native or engineered CRISPR enzymes, namely SaCas9, SpCas9_VQR, SpCas9_EQR, SpCas9_VRER and AsCpf1, were analyzed. SaCas9 is a Cas9 orthologue from Staphylococcus aureus (Ran, et al. (2015) Nature 520:186-191). SpCas9_VQR, SpCas9_EQR and SpCas9_VRER are engineered SpCas9 variants from Streptococcus pyogenes. AsCpf1 is a class 2 CRISPR enzyme from Acidaminococcus which is highly specific for a T-rich motif (TTTN). Only highly effective PAMs recognized by these enzymes were analyzed (Table 5). A SNP can mediate the loss, gain, or loss/gain of PAMs and may induce or destroy PAMs for multiple enzymes. Two types of SNPs can mediate the deletion of the mHTT: 1) SNPs causing loss of a PAM in the normal chromosome; 2) SNPs causing gain of a PAM in the mHTT chromosome. The effect of the 19 SNPs on different PAMs, and their AF, is shown in FIG. 4E.

Among them, SNPs1-6 are identical to the six SNPs identified and tested (Monteys, et al. (2017) Mol. Ther. 25:12-23). All have much higher AFs in the normal chromosomes than in the mHTT chromosomes (Table 1, FIG. 4B). For example, the AF of SNP1 in the normal chromosomes is 23.51%, which is 8.3 times of the AF in the mHTT chromosomes (2.82%). All six SNPs cause PAM loss. Thus, the bias in AF (higher abundances on the normal chromosomes) allows HD individuals to benefit from this genome editing strategy. This is consistent with the observations that SNP1 mediated deletion of the expanded HTT in 9 of 11 cell lines (Table S4 of Monteys, et al. (2017) Mol. Ther. 25:12-23).

Sixteen of the 19 SNPs cause the loss or gain of at least one PAM. In addition to SNPs1-6, SNP16 and SNP17 mediate PAM loss in the normal allele, with an AF >30% in the normal chromosomes. The AFs of SNP16 in normal and mHTT chromosomes are 36.99% and 11.6%, respectively. The alternative allele of SNP16 disrupts the PAM of SpCas9_VQR and SpCas9_EQR (ref: C[G]AG; alt: C[A]AG). Both SpCas9_VQR and SpCas9_EQR recognize the PAM of NGAG but SpCas9_VQR also recognizes the motif of NAAG, at lower efficiency (Kleinstiver, et al. (2015) Nature 523:481-485). SNP16 can also be targeted by SaCas9 (ref: TC[G]AGT; alt: TC[A]AGT). SNP17 resides in a ploy-T region and exists in 31.03% of normal chromosomes assessed, and in 7.84% of mHTT chromosomes. SNP17 disrupts the PAM of AsCpf1 (ref: TT[T]T; alt: TT[C]T), which has a strong selectivity for the TTTN PAMs and thus SNP17 is AsCpf1 specific.

The specificity of the guide RNA sequence is critical for preventing off-target cleavage events. For the Cas9 enzyme and its variants, the guide sequence is on the 5′ side of the PAM. But for the Cpf1 enzymes, the guide sequence is on the 3′ side of the PAM. The Benchling website (benchling.com) was used to design guide RNAs and predict their specificity. The guide RNA sequence for SNP16 is very specific with an off-target score of 98.4 (score ranging in 0-100, the higher the better). The guide RNA sequence (TAAAAATAAAAATAAGTTAACAC; SEQ ID NO: 140) for SNP17 is not specific, with an off-target score of 43.3. This sequence contains a poly-A and may have multiple copies in the human genome. Therefore, SNP16 is a strong candidate with high AF and specificity.

SNP16 genotyping was validated from long reads using two traditional methods. First, Sanger sequencing primers were designed for SNP16 and randomly sequenced 20 samples from plate-1 of the French cohort. The Sanger sequencing results were completely consistent with the SNP genotypes called from the nanopore long reads. TaqMan® real-time PCR assay was also used to genotype three 96-well plates (288 samples) of the CHDI cohort. The TaqMan® assay results were also completely consistent with the SNPs called from the nanopore long reads. This indicates that the SNP calls made from the nanopore long reads are of high accuracy.

### Experimental Validation of the Allele-Specific Cleavage Mediated by SNP16

The screen identified SNP16 (rs3856973) as a novel prevalent SNP within HTT intron-1 that could be used alone or together with SNP1 (rs2857935) to edit and terminate mHTT expression. sgRNA sequences targeting SNP16 (sgHD16) and SNP1 (sgHD1) were designed to mediate SaCas9 editing of the mHTT exon-1. The sgHDi3 (targeting intron-1) and sgCtl guides (Monteys, et al. (2017) Mol. Ther., 25:12-23) were modified to complex with SaCas9. The editing efficacy of three pairs were tested: sgHD1/16 (targeting SNP1 and SNP16), sgHD1/13 (positive control), and sgCtrl (negative control) (FIG. 5A). HEK293 cells, which are homozygous for both SNP1 and SNP16, were transfected with sgRNA/SaCas9 expression plasmids and genomic deletions were assessed. DNA products of the anticipated size were amplified in all sgRNA/SaCas9 pairs tested. The HTT genomic locus remained intact on cells co-expressing SaCas9 and the sgCtl (negative control group), whereas a band resulting from HTT exon-1 deletion was observed on cells transfected with the sgHD1/13 SaCas9 or the sgHD1/16 SaCas9 cassettes (FIG. 5B). Notably, the intensity of the amplified DNA bands indicates that the editing efficacy of sgHD1/i3 was higher than the sgHD1/16 pair. HTT mRNA levels were reduced in cells following editing, as determined by qPCR (FIG. 5C). Reduction of HTT mRNA levels was greater in cells expressing sgHD1/i3 than the sgHD1/16 pairs, mirroring what was observed by PCR of genomic DNA.

Next, it was tested if the sgHD1/16 pair can be used for expanded allele-specific targeting. The ND33392 fibroblast line contains heterozygous SNP1 and SNP16 with the SNP-dependent PAMs on the mHTT allele (FIG. 5D). PCR amplification of genomic DNA using primer pairs binding within the mHTT exon-1 sequence showed targeted cleavage of the mHTT allele in cells electroporated with plasmids expressing sgHD1/13/SaCas9 and sgHD1/16/SaCas9 relative to those electroporated with the control sgCtl/SaCas9 complex (FIGS. 5E-5F).

### Analysis of Haplotypes of the HD Samples

To better understand the percent of HD individuals that could benefit from this editing approach, the haplotypes of normal and mHTT chromosomes of the HD individuals as well as those in the 1000 Genomes Project phase 3 data set (denoted as 1 KG data set hereafter) were analyzed. The 1 KG data set provides phased SNP/Indel calls of 2,504 individuals, of which 404 individuals are from the NFE population. In the following haplotype analysis, all SNPs listed in FIG. 4E were used except SNP11, as it resides in the CAG-CCG repeat region and was not genotyped in the 1 KG data set.

In total, 37 different haplotypes were observed in the French HD cohort (including normal and mHTT chromosomes) and individuals of NFE population in the phase 3 data set. The frequencies of the top 10 most abundant haplotypes are shown in FIG. 6A. Hap1 (no SNP across the region) is the predominant haplotype (82.58%) in the mHTT chromosomes. It is also the most abundant haplotype in normal chromosomes (frequency=48.39%). The frequencies of minor haplotypes (haplotypes 2-37) are dramatically different between normal and mHTT chromosomes. The frequencies of the minor haplotypes in the normal chromosomes are highly correlated (R²=0.8036) with those in the 1 KG data set. In contrast, the haplotype frequencies in the mHTT chromosomes have no correlation (R²=0.0031) with those in the 1 KG data set (FIGS. 6C-6D). To validate these discoveries, the haplotypes of the CHDI cohort were examined, which contains HD samples from multiple continents and ethnicities. Similar to the French cohort, Hap1 is the predominant haplotype (>80%) of the mHTT chromosomes, in samples from all continents and ethnic groups with more than 50 samples (FIGS. 6E-6F). It was also tested if there is an association between the expanded CAG repeats and haplotypes. The median CAG repeat size is between 45 and 48 in all groups (FIG. 6B). A haplotype that is associated with specific repeat sizes was not found.

Since the haplotypes of all HD individuals have been identified, it is possible to estimate the percentage of people potentially amenable to the allele-specific genome editing strategy (FIG. 6G); 22% of the HD individuals can be edited at SNP1 and 30.2% of the HD individuals can be edited at SNP16. The fraction of HD subjects who have SNP16 on both mutant and normal HTT, mutant HTT only, normal HTT only, and none are 4.9%, 7.2%, 30.2% and 57.8%, respectively. A combination of SNPs1-6 and SNP16 can edit 41% HD individuals. Up to 57% HD individuals can be edited if all SNPs are targeted. However, 43% HD individuals cannot be edited as 91% of them carry homozygous hap1 and there is no heterozygous SNP in the region covered by the sequencing data (5073 bp upstream and 4884 bp downstream of exon-1).

Searching for Potential Editing Sites for Individuals with Hap1 in Both Alleles

Additional strategies were tried to find potential editing sites for individuals with hap1 in both alleles. First, Indels and structural variants (SVs) were called from the sequencing data. However, all common Indels/SVs are located in tandem repeat regions where the guide RNAs are not specific. Next, SNPs were analyzed within a greater range than the 10 kb limit using the 1 KG data set (NFE population). Since the 1 KG data set provides phased SNPs, the SNPs from chromosome 4 with different haplotypes can be extracted. It was found that in hap1 chromosomes, there are fewer SNPs in the HTT gene (167 kb) compared with the intergenic region. In regions upstream of the HTT gene, the nearest high-frequency SNP (AF>20%) in hap1 chromosomes is 12.4 kb away from exon-1, which is outside of the range useful for efficient editing. In the region downstream of exon-1, the closest high-frequency SNP is 20.7 kb away, similarly of limited utility for deleting exon-1. However, this SNP resides close to exon-3 and could be a candidate site to delete exon-3. Several high-frequency SNPs close to exon-6, exon-17, exon-23, and others were also found. The HTT gene is a large gene with 67 exons. Theoretically, deletion of exon-3/exon-6/exon-23 would shift the reading frame and cause a premature stop codon disrupting huntingtin protein expression. However, if expanded exon-1 is left intact, alternative splicing events or RNA translation could still occur (Sathasivam, et al. (2013) Proc. Natl. Acad. Sci., 110:2366-2370; Banez-Coronel, et al. (2015) Neuron 88:667-677; Neueder, et al. (2017) Sci. Rep., 7:1307).

Herein, a targeted long-read sequencing approach was developed to resolve a 10.4 kb genomic region flanking the CAG repeats in exon-1 of the HTT gene. This approach was applied to two independent HD cohorts for the purpose of allele specific editing for HD therapeutic development. Genomic variants from the sequencing data were called and potential gene-editing sites that could mediate allele-specific deletions of the mHTT allele systematically analyzed. The results showed that 22% HD individuals can be edited by targeting SNP1 (Monteys, et al. (2017) Mol. Ther., 25:12-23). In addition, SNP16 was identified as a novel candidate, which can target more people (30%) than SNP1 does in HD individuals of European ancestry. In proof-of-concept experiments, gRNAs targeting SNP16 could effectively edit mHTT cell lines. Overall, the haplotype analysis reveals that up to 57% HD individuals of European ancestry can be targeted in an allele-specific manner by combinatorial editing.

Hap1 is the predominant haplotype in both normal chromosomes (48.39%) and mHTT chromosomes (82.58%). Due to the lack of diversity in the haplotypes, about 40% HD individuals carry hap1 in both alleles and cannot be edited by targeting a SNP near exon-1. Unfortunately, further analyses using other strategies did not find high-frequency indels or SVs that could mediate allele-specific editing; the nearest high-frequency SNP was at least 10 kb away. Despite this, deletion of other exons in an allele-specific manner is possible. Their deletion could induce an open reading frame shift causing, a premature stop codon, and loss of mHTT expression. However, toxicity from exon-1-derived transcripts would remain (Sathasivam, et al. (2013) Proc. Natl. Acad. Sci., 110:2366-2370; Banez-Coronel, et al. (2015) Neuron 88:667-677; Neueder, et al. (2017) Sci. Rep., 7:1307).

SNPs and HD haplotypes have been analyzed (Kay, et al. (2019) Am. J. Hum. Genet., 105:1112-1125; Kay, et al. (2015) Mol. Ther., 23:1759-1771; Chao, et al. (2017) Eur. J. Hum. Genet., 25:1202-1209). For this, SNP arrays were used to genotype common SNPs and focused on target sites for allele-specific knockdown by ASOs or RNAi. Thus the haplotypes were based on the HTT gene, most of which are distant from exon-1. Here, long-read sequencing was used. It has the advantage over SNP array genotyping and short-read sequencing in that it can perform repeat quantification, SNP detection and haplotyping at the same time. With long reads, the haplotyping process is straightforward and does not require trio data. It was possible to assemble the diploid genome sequence for each HD individual. Cumulatively, the data provide a comprehensive analysis of allele-specific target sites for CRISPR-based gene editing, which relies on the ability of a SNP to provide a PAM site for targeted editing of the expanded allele. For effective editing, genomic regions within 5 kb of exon-1 were focused on, because the distance between upstream and downstream guides influenced editing efficacy (Monteys, et al. (2017) Mol. Ther., 25:12-23).

Long reads have a higher per-base error rate than short reads. However, the sequencing error tends to be random and the consensus sequence of high-coverage long reads can be very accurate (Wick, et al. (2019) Genome Biol., 20:129). At least 50× coverage per allele was required for analysis, and the vast majority of samples have more than 200× coverage per allele. In the results, the allele frequencies of the SNPs in normal chromosomes are highly correlated with those in the gnomAD database, indicating the SNP detection is correct. The haplotype frequencies in normal chromosomes are also highly correlated with those in the 1 KG data set. Additionally, the genotypes of SNP16 was validated using Sanger sequencing and a TaqMan® SNP genotyping assay, both of which generated results identical to the Oxford Nanopore long reads. Of note, Oxford Nanopore sequencing produces more indel errors in homopolymer regions (e.g., polyA sequence). Therefore, Indel detection from Nanopore reads may be less accurate in homopolymer regions. As this study focuses on SNP detection, the data are less affected by this limitation.

NanoBinner and NanoRepeat are novel computational tools developed for this work. NanoBinner is a demultiplexer for amplicon sequencing data. Existing tools such as DeepBinner (Wick, et al. (2018) PLOS Comput. Biol., 14: e1006583) and qcat (github.com/nanoporetech/qcat) only support the barcoding kits provided by Oxford Nanopore Technologies (ONT). NanoBinner is a general tool and can work with any user-provided barcodes. NanoRepeat is a tool for repeat detection from amplicon sequencing. It uses alignment-based quantification and can jointly quantify two adjacent tandem repeats, phase the reads and report haplotypes. NanoRepeat was evaluated on both whole-genome sequencing data and amplicon sequencing data of the HD cohort. In the whole-genome data, NanoRepeat outperformed other repeat quantification tools and reduced the average quantification error by 3.5-fold. In the amplicon sequencing data of the CHDI cohort, NanoRepeat is highly consistent with PCR-based fragment analysis, which is commonly used in clinical labs for diagnostic purposes.

sgRNAs targeting SNPs1-6 were validated (Monteys, et al. (2017) Mol. Ther., 25:12-23). In this study, sgRNAs targeting SNP16 were developed and tested it in cell lines. There was significant reduction of HTT levels in HEK293 cells and mHTT in ND33392 fibroblast cells. However, the efficacy of editing SNP1/16 (two SNPs) was not higher than editing SNP1/13 (one SNP and one homozygous site), which may due to the longer distance between the SNP1/16 sgRNA/Cas9 complexes and sequence context at SNP16. Nonetheless, SNP16 is an effective candidate because it creates a PAM more frequently than SNP1 and editing at SNP16 would benefit HD individuals without SNP1.

In summary, an experimental and computational workflow was developed to resolve the SNP haplotypes near exon-1 of the HTT gene for allele specific editing. This workflow was applied to two HD cohorts and comprehensively analyzed potential sites for allele-specific deletion of mHTT for CRISPR/Cas systems. A detailed haplotype map for the region near HTT exon-1 was developed, which may be applied to other editing strategies and newly emerging editing enzymes. Additionally, the workflow and novel computational tools can be applied to other repeat expansion disorders.

TABLE 6
SNPs in chromosome 4.

| Position | | | |
|---|---|---|---|
| (GRCh38) | Ref | Alt | RS_number |
| 3069663 | A | G | rs115297890 |
| 3069717 | C | T | rs941395326 |
| 3069914 | A | G | N.A. |
| 3069930 | T | C | rs536580405 |
| 3069952 | C | G | rs35631490 |
| 3070075 | A | G | rs73086139 |
| 3070120 | C | G | rs73086140 |
| 3070354 | G | A | rs190434238 |
| 3070451 | C | T | rs61791259 |
| 3070504 | G | A | rs61792460 |
| 3070599 | G | A | rs73086144 |
| 3070636 | C | G | rs73086145 |
| 3070672 | A | G | rs61792461 |
| 3070714 | C | G | rs61792462 |
| 3071145 | T | A | N.A. |
| 3071198 | C | G | N.A. |
| 3071275 | A | C | rs74444018 |
| 3071436 | A | G | rs61090955 |
| 3071526 | C | T | rs77384845 |
| 3071527 | A | G | rs10011412 |
| 3071584 | C | T | rs536384294 |
| 3071629 | T | A | rs10014333 |
| 3071658 | G | C | rs61792464 |
| 3071780 | C | T | rs79649118 |
| 3071980 | A | G | rs1712184015 |
| 3072190 | G | A | N.A. |
| 3072237 | C | T | rs78912730 |
| 3072319 | T | C | N.A. |
| 3072376 | G | T | rs112435590 |
| 3072746 | G | A | rs111670395 |
| 3072781 | C | A | rs539378701 |
| 3072816 | C | T | rs183207300 |
| 3072974 | G | T | rs187490343 |
| 3073068 | A | G | rs762855 |
| 3073238 | C | G | rs9996199 |
| 3073268 | G | A | rs914303630 |
| 3073403 | C | T | N.A. |
| 3073814 | G | T | rs115117610 |
| 3073861 | T | C | rs28431418 |
| 3073964 | G | C | rs2857935 |
| 3073964 | G | T | rs2857935 |
| 3073976 | A | G | rs868433409 |
| 3074009 | C | T | rs28616835 |
| 3074104 | C | G | rs1032674750 |
| 3074156 | T | C | rs1215098751 |
| 3074157 | A | G | N.A. |
| 3074301 | T | G | rs149624523 |
| 3074301 | T | C | rs149624523 |
| 3074349 | T | C | rs192641280 |
| 3074423 | C | A | rs112396951 |
| 3074454 | C | G | rs13122415 |
| 3074455 | C | T | rs189174671 |
| 3074539 | A | G | rs13132932 |
| 3074610 | C | A | rs1288198096 |
| 3074663 | A | G | rs918190068 |
| 3074678 | G | A | rs13102260 |
| 3074723 | C | T | rs146151652 |
| 3074927 | G | A | rs779821751 |
| 3074932 | A | C | rs746852037 |
| 3074935 | A | C | rs61792465 |
| 3074936 | A | G | rs473915 |
| 3074938 | A | C | rs61792466 |
| 3074945 | A | G | rs76533208 |
| 3075082 | A | C | rs868348450 |
| 3075228 | A | C | rs10006977 |
| 3075230 | G | A | rs76525526 |
| 3075310 | T | C | rs10009935 |
| 3075522 | A | T | rs77845883 |
| 3075656 | G | A | rs879677039 |
| 3075679 | A | G | rs866416034 |
| 3075942 | A | G | rs933735047 |
| 3076029 | A | T | rs34045730 |
| 3076049 | G | A | rs73191179 |
| 3076208 | C | A | N.A. |
| 3076258 | T | C | rs28656215 |
| 3076279 | G | C | rs28571971 |
| 3076280 | T | C | rs28583447 |
| 3076320 | C | G | rs28468636 |
| 3076335 | C | A | rs28564368 |
| 3076364 | G | A | rs28485764 |
| 3076434 | A | G | rs146187080 |
| 3076455 | C | T | rs762525632 |
| 3076520 | A | G | rs77173925 |
| 3076616 | G | T | rs1336317078 |
| 3076952 | C | T | rs544808684 |
| 3077210 | A | G | rs3905238 |
| 3077251 | A | G | rs186362196 |
| 3077339 | A | T | rs527605641 |
| 3077387 | A | G | rs902399863 |
| 3077697 | T | C | rs549749709 |
| 3077715 | G | C | rs186788713 |
| 3077840 | G | T | N.A. |
| 3078179 | G | C | rs28377140 |
| 3078235 | C | T | rs554364238 |
| 3078446 | G | A | rs3856973 |
| 3078472 | A | G | rs4498089 |
| 3078511 | T | C | rs12508079 |
| 3078541 | C | T | rs1285517229 |
| 3078688 | C | T | rs148125464 |
| 3078730 | G | C | rs112527131 |
| 3078786 | G | C | rs1456128915 |
| 3078835 | C | T | rs57666989 |
| 3078978 | G | C | rs10006129 |
| 3079083 | A | G | rs28696693 |
| 3079165 | G | C | rs28755900 |
| 3079762 | A | G | rs113674085 |
| 3079780 | C | A | rs933221275 |
| 3079781 | C | T | rs144376040 |
| 3079783 | G | T | rs913163390 |
| 3079915 | A | G | rs28393280 |

A number of publications and patent documents are cited throughout the foregoing specification in order to describe the state of the art to which this invention pertains. The entire disclosure of each of these citations is incorporated by reference herein.

While certain of the preferred embodiments of the present invention have been described and specifically exemplified above, it is not intended that the invention be limited to such embodiments. Various modifications may be made thereto without departing from the scope and spirit of the present invention, as set forth in the following claims.

1. A method for inhibiting, treating, and/or preventing Huntington's disorder in a subject in need thereof, said method comprising reducing the expression of a mutant protein encoded by an allele of the huntingtin (HTT) gene associated with the Huntington's disorder in said subject,
wherein said method comprises administering to the subject a nucleic acid molecule encoding Cas9 and at least two guide RNAs.

2. A method for reducing the expression of a mutant protein encoded by an allele of the huntingtin (HTT) gene associated with Huntington's disorder in a cell, said method comprising delivering to the cell a nucleic acid molecule encoding Cas9 and at least two guide RNAs.

3. The method of claim 1, wherein said method comprises administering to the subject or cell a nucleic acid molecule encoding Cas9, at least one guide RNA, and, optionally at least one donor DNA.

4. The method of claim 1, wherein said guide RNA are administered as a nucleic acid molecule encoding said guide RNA.

5. The method of claim 4, wherein said nucleic acid molecules are administered in an expression vector.

6. The method of claim 5, wherein said expression vector is a viral vector.

7. The method of claim 1, wherein at least one guide RNA targets a sequence adjacent to a PAM present on only one allele of the gene.

8. The method of claim 7, wherein said PAM is present only on the mutant allele.

9. The method of claim 1, wherein said at least two guide RNAs target a sequence adjacent to a PAM present on only one allele of the gene.

10. The method of claim 1, wherein a first guide RNA targets a sequence within the promoter or 5′ untranslated region of the gene and a second guide RNA targets a sequence within the first intron of the gene.

11. The method of claim 10, wherein the first guide RNA targets SNP1 and the second guide RNA targets SNP16.

12. The method of claim 1, wherein at least one guide RNA targets SNP16.

13. The method of claim 1, wherein at least one guide RNA targets SNP1, at least one guide RNA targets SNP2, at least one guide RNA targets SNP3, at least one guide RNA targets SNP4, at least one guide RNA targets SNP5, at least one guide RNA targets SNP6, and at least one guide RNA targets SNP16.

14. The method of claim 1, wherein at least one guide RNA comprises GATAGGGAAATGTCAGGGTTAA (SEQ ID NO: 134).

15. An isolated guide RNA, wherein said guide RNA targets a sequence adjacent to a PAM present on only one allele of a gene associated with a polyglutamine disorder.

16. A nucleic acid molecule encoding at least one of the guide RNA of claim 15.

17. A vector comprising the nucleic acid molecule of claim 16.

18. The vector of claim 17, which is a viral vector.

19. The vector of claim 17, further comprising a nucleic acid molecule encoding Cas9.

20. A composition comprising at least one nucleic acid molecule of claim 16 and a carrier.
